(19) United States
(12) Patent Application Publication
(10) Pub. No.: US 2016/0177222 A1
(43) Pub. Date: Jun. 23, 2016

(54) IONIC LIQUID SYSTEMS

(71) Applicant: The Procter & Gamble Company, Cincinnati, OH (US)

(72) Inventors: Giulia Ottavia BIANCHETTI, Rome (IT); Sarah Anne TOZER, London (GB); Pauline Mary MCNAMEE, Camberley (GB); Lynette Anne Makins HOLLAND, Abbots Langley (GB); Kenneth Richard SEDDON, Belfast (GB); Harambage Quintus Nimal GUNARATNE, Belfast (GB); Shane Declan DEVINE, Belfast (GB); Julien ESTAGER, Belfast (GB); Feng-Ling WU, Belfast (GB); Alberto Vaca PUGA, Belfast (GB)

(21) Appl. No.: 14/964,631

(22) Filed: Dec. 10, 2015

Related U.S. Application Data
(60) Provisional application No. 62/094,070, filed on Dec. 19, 2014.
(60) Provisional application No. 62/115,149, filed on Feb. 12, 2015.

(51) Int. Cl. C11B 9/00; A61K 8/35; A61K 8/46; A61K 8/41; A61Q 5/02; A61Q 15/00; C07C 211/63; C07C 47/575; C07C 309/17; A61Q 5/12; A61Q 19/00; C11D 3/50; A61Q 19/10

(52) U.S. Cl. CPC C11B 9/0061; C11D 3/50; A61K 8/35; A61K 8/466; A61K 8/416; A61Q 5/02; A61Q 19/10; C07C 211/63; C07C 47/575; C07C 309/17; C11B 9/0011; A61Q 5/12; A61Q 19/00; A61Q 15/00

(57) ABSTRACT

The present invention relates to an ionic liquid system for enhanced delivery and/or deposition of a perfume raw material onto a substrate, particularly fabric, hard surfaces, soft surfaces, skin, or hair. The invention also relates to consumer products comprising the new ionic liquid systems, and processes for making and methods of using such ionic liquid systems and consumer products.

### FIELD OF THE INVENTION

The present invention relates to ionic liquid systems and consumer products comprising such ionic liquid systems, as well as processes for making and using thereof.

### BACKGROUND OF THE INVENTION

Consumer products may comprise one or more perfume systems that can deposit a desired fragrance upon a substrate that is contacted with such a product. Current perfume systems, such as for example perfume micro-capsule (“PMC”) technology, encapsulates perfume raw materials (“PRMs”). The PMC comprises a shell wall material and a core material of PRMs that is encapsulated within the shell wall material. The PMC can enable deposition on substrate and release over-time of the PRMs contained therein, for example by breakage of the micro-capsules (from mechanical stress such as friction, i.e., friable) or leakage of the PRMs out of the PMC and onto the substrate. However, it has been noticed that several PRMs having low c Log P values, preferably below 3, tend to be hydrophilic and pre-maturely leak out of the PMC during storage. As a result, these PRMs are lost before they have an opportunity to be delivered and/or deposited onto the substrate to provide the desired scent. Furthermore, malodour removal/control is an important consumer benefit. Consumer products commonly incorporate PRMs in their formulation to aid in masking malodour. If certain PRMs are pre-maturely lost then they will not have an opportunity to provide their anti-malodour benefits as well.

Recently, ionic liquids (“ILs”) have been used in the fragrance industry for dealing with solvent applications of the synthesis of fragrance materials or with the extractions of naturally derived PRMs (Sullivan, N., Innovations in Pharma. Tech. 2006, 20:75-77). For example, Forsyth et al. investigated the utilisation of ionic liquid solvents for the synthesis of lily-of-the-valley fragrance and fragrance intermediate Lilial (Forsyth et al., J. Mol. Cat. A. 2005, 231:61-66). Additionally, the utilisation of ionic liquids to suppress evaporation of fragrances in consumer products has also been gaining attention (Davey P., Perfumer Flavorist 2008, 33(4):34-35). For instance, ionic liquids have been used as “fixatives” with fragrance compositions to delay the rate of evaporation of the perfume component to impart increased stability/longevity of the fragrance (Petrat et al., US2006/0166856). Ionic liquids have also been used as pro-fragrances where PRM is appended covalently to either the cation or the anion (Rogers et al., US2012/046244; Blesic et al., RSC Advances, 2013, 3:329-333).

Accordingly, as discussed above, the prior art efforts have focussed on the incorporation of ionic liquids into an existing fragrance composition whereby the ionic liquids associate with the PRMs via various mechanism, such as for example electrostatic interactions/hydrogen-bonding non-covalent forces. The prior art does not appear to focus on cations and/or anions derived from perfume raw materials that form the ionic liquids. The prior art also does not appear to focus on using ionic liquids for improving the deposition of fragrances onto substrates and/or delivering other benefits (e.g., freshness delivery, biofilm removal, or anti-malodour). Applicants have surprisingly discovered that for ionic liquid systems comprising cations and anions, the conjugate acids of the anions can be derived from certain PRMs with suitable pKₐ and that can release the perfume raw materials upon drying out after deposition onto a substrate. Therefore, there remains a need for an ionic liquid system that comprises ionic liquids to deliver and/or deposit fragrances onto a substrate, preferably leveraging low c Log P PRMs. It is also advantageous that the ionic liquids in the ionic liquid system aids in freshness delivery, biofilm removal and/or malodour control. It is also a further advantage of the invention to maximize cost/efficiency benefits by using an anion, and its conjugate acid, with dual functions.

### SUMMARY OF THE INVENTION

In a first aspect, the present invention is directed to an ionic liquid system comprising one or more ionic liquids, each comprising of a cation and an anion, wherein the conjugate acid of at least one of the the anions is a perfume raw material with a pKₐ of from about 0 to about 14, preferably from about 0 to about 8, or more preferably from about 4 to about 8.

In another aspect of the present invention, a consumer product comprising from about 0.0001% w/w to 100% w/w, based on total consumer product weight, of an ionic liquid system as disclosed herein.

In still another aspect of the present invention, a method of delivering and/or depositing fragrance onto a fabric comprising the steps of optionally washing and/or rinsing the fabric, contacting the fabric with a detergent composition as disclosed herein, then optionally washing and/or rinsing the fabric.

These, and other features of the present invention, will become apparent to one skilled in the art upon review of the following detailed description when taken in conjunction with the appended claims.

### DETAILED DESCRIPTION OF THE INVENTION

### Definitions

As used herein, articles such as “a” and “an” when used in a claim, are understood to mean one or more of what is claimed or described.

As used herein, the terms “include”, “includes” and “including” are meant to be non-limiting.

As used herein, the term “composition” or “consumer product” are used interchangeably, those compositions intended for the treatment of hard surfaces (e.g., floors, countertops, sinks, windows, toilets, dishware), soft surfaces (e.g., carpets, fabric), air (e.g., air fresheners, fabric refresheners), skin and hair (e.g., shampoos, body wash, shave care) including products, packaging or devices generally intended to be used or consumed in the form in which it is sold. Such products include but are not limited to products for and/or methods relating to treating fabrics, dishes, air care including air freshners and scent delivery systems, car care, dishwashing, fabric conditioning (including softening and/or freshening), laundry detergency, laundry and rinse additives and/or care, hard surface cleaning and/or treatment including floor and toilet bowl cleaners, and other cleaning for consumer or institutitional use.

As used herein, the term “detergent composition” is a subset of consumer products that includes, unless otherwise indicated, include a surfactant. Non-limiting examples of detergent compositions include: detergents, laundry detergents, fabric softeners, and laundry additives. The detergent composition of the present invention may be used for handwashing, administered to an automated laundry washing machine as well as for soaking and/or pre-treating fabrics. The detergent composition may be in the form of a powder/granule, a bar, a pastille, foam, flakes, a liquid, a dispersible substrate, or as a coating on a dryer added fabric softener sheet. The detergent composition may be administered to the washing machine as a unit dose or dispensed from a container (e.g., dispensing cap) containing multiple doses. An example of a unit dose is a composition encased in a water soluble polyvinylalcohol film. All of such products which are applicable may be in standard, concentrated or even highly concentrated form even to the extent that such products may in certain aspect be non-aqueous.

As used herein, the term “fragrance profile” means the description of how the fragrance is perceived by the typical human nose after it has been applied to a substrate. It is a result of the combination of the PRMs, if present, of a consumer product. A fragrance profile is composed of 2 characteristics: ‘intensity’ and ‘character’. The ‘intensity’ relates to the perceived strength whilst ‘character’ refers to the odour impression or quality of the perfume, i.e., fruity, floral, woody, etc.

As used herein, the term “perfume raw material” (“PRM”) and “perfume raw materials” (“PRMs”) relates to a perfume raw material, or a mixture of perfume raw materials, that are used to deliver and/or deposit an overall pleasant odour or fragrance profile to a consumer product or a substrate upon which the consumer product is applied. “Perfume raw materials” can encompass any suitable perfume raw materials for fragrance uses, including materials such as, for example, alcohols, aldehydes, ketones, esters, ethers, acetates, nitriles, terpene hydrocarbons, nitrogenous or sulfurous heterocyclic compounds and essential oils. However, naturally occurring plant and animal oils and exudates comprising complex mixtures of various chemical components are also know for use as PRMs. The individual PRMs which comprise a known natural oil can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, N.J., USA and more recently re-publisehd by Allured Publishing Corporation Illinois (1994). Additionally, some PRMs are supplied by the fragrance houses (e.g., Firmenich, International Flavors & Fragrances, Givaudan, Symrise) as mixtures in the form of proprietary speciality accords. Non-limiting examples of the PRMs useful herein include pro-fragrances such as acetal pro-fragrances, ketal pro-fragrances, ester pro-fragrances, hydrolisable inorganic-organic pro-fragrances, and mixtures thereof. The PRMs may be released from the pro-fragrances in a number of ways. For example, the fragrance may be released as a result of simple hydrolysis, or by a shift in an equilibrium reaction, or by a pH-change, or by enzymatic release or by thermal change or by photo-chemical release.

As used herein, the term “perfume system” or “perfume composition” can be used interchangeably and refers to the component in the consumer product composition or ionic liquid system that is formed of PRMs, i.e., ingredients capable of imparting or modifying the odour of the consumer product itself or the substrate to which it is applied.

As used herein, the term “substrate” includes for non-limiting example, fabrics, garments, hard surfaces, soft surfaces, dishware, hair and body, etc.

Unless otherwise noted, all component or composition levels are in reference to the active level of that component or composition, and are exclusive of impurities, for example, residual solvents or by-products, which may be present in commercially available sources. All percentages and ratios are calculated by weight unless otherwise indicated. All percentages and ratios are calculated based on the total weight of the consumer product, which includes the product and product matrix composition unless otherwise indicated.

In all embodiments of the present invention, all percentages are by weight of the total fragrance composition, as evident by the context, unless specifically stated otherwise. All ratios are weight ratios, unless specifically stated otherwise, and all measurements are made at 25° C., unless otherwise designated.

Certain chemical functional groups named here are preceded by a shorthand notation indicating the total number of carbon atoms that are to be found in the indicated chemical group. For example: C₁-C₂₀ alkyl describes an alkyl group having a total of 1 to 20 carbon atoms (e.g. C₁₀ implies C₁₀H₂₁). The total number of carbons in the shorthand notation does not include carbons that may exist in substituents of the group described. Unless specified to the contrary, the following terms have the following meaning:

“Alkyl” refers to a group containing a straight or branched hydrocarbon chain consisting solely of carbon and hydrogen atoms, containing no unsaturation, having from 1 to 20 carbon atoms, preferably 1 to 12 carbon atoms, preferably 1 to 8, or preferably 1 to 6 carbon atoms, and which is attached to the rest of the molecule by a single bond, e.g., methyl, ethyl, propyl, 1-methylethyl (iso-propyl), butyl, pentyl, and the like. An alkyl may be optionally substituted.

“Alkenyl” refers to a group containing straight or branched hydrocarbon chain consisting solely of carbon and hydrogen atoms, containing at least one carbon-carbon double bond, having from 2 to 20 carbon atoms, preferably 2 to 12 carbon atoms, or preferably 1 to 8 carbon atoms, e.g., ethenyl, prop-2-enyl, but-1-enyl, pent-1-enyl, penta-1,4-dienyl, and the like. An alkenyl may be optionally substituted.

“Alkynyl” refers to a group containing straight or branched hydrocarbon chain consisting solely of carbon and hydrogen atoms, containing at least one carbon-carbon triple bond, having from 2 to 20 carbon atoms, preferably 2 to 12 carbon atoms, or preferably 1 to 8 carbon atoms, e.g., ethynyl, propynyl, butynyl, pentynyl, hexynyl, and the like. An alkynyl may be optionally substituted.

“Alkylene” or “alkylene chain” refers to a group containing straight or branched hydrocarbon chain linking the rest of the molecule to a group, consisting solely of carbon and hydrogen, containing no unsaturation and having from 1 to 12 carbon atoms, e.g., methylene, ethylene, propylene, butylene, and the like. An alkylene may be optionally substituted.

“Alkenylene” or alkenylene chain” refers to a straight or branched hydrocarbon chain linking the rest of the molecule to a group, consisting solely of carbon and hydrogen, containing at least one carbon-carbon double bond and having from 2 to 20 carbon atoms, preferably 2 to 12 carbon atoms, e.g., ethenylene, propenylene, butenylene, and the like. An alkenylene may be optionally substituted.

“Alkynylene” or “alkynylene chain” refers to a straight or branched hydrocarbon chain linking the rest of the molecule to a group, consisting solely of carbon and hydrogen, containing at least one carbon-carbon triple bond and having from 2 to 20 carbon atoms, e.g., propynylene, butynylene, and the like. An alkynylene may be optionally substituted.

“Alkoxy” refers to a functional group of the formula —ORₐ where Rₐ is an alkyl chain as defined above containing 1 to 20 carbon atoms, preferably 1 to 12 carbon atoms. An alkoxy may be optionally substituted.

“Alkoxyalkyl” refers to a functional group of the formula —Rₐ₁—O—Rₐ₂ where Rₐ₁ j is an alkylene as defined above and Rₐ₂ is an alkyl chain as defined above containing 1 to 20 carbon atoms, preferably 1 to 12 carbon atoms. An alkoxyalkyl may be optionally substituted.

“Aryl” refers to aromatic monocyclic or multicyclic hydrocarbon ring system consisting only of hydrogen and carbon, and preferably containing from 6 to 18 carbon atoms, preferably 6 to 10 carbon atoms, where the ring system is aromatic (by the Hückel definition). Aryl groups include but are not limited to groups such as phenyl, naphthyl, anthracenyl. The term “aryl” or the prefix “ar” (such as in “aralkyl”) is meant to include aryls that may be optionally substituted.

“Cycloalkyl” refers to a stable saturated mono-cyclic or polycyclic hydrocarbon group consisting solely of carbon and hydrogen atoms, which may include fused or bridged ring systems, having from 3 to 15 carbon atoms, preferably having from 3 to 10 carbon atoms or preferably from 3 to 7 carbon atoms. A cycloalkyl may be optionally substituted.

“Cycloalkylalkyl” refers to a functional group of the formula —RₐR_{d}, where Rₐ is an alkylene as defined above and R_{d} is a cycloalkyl as defined above.

“Haloalkyl” refers to an alkyl as defined above that is substituted by one or more halogen groups, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A haloalkyl may be optionally substituted.

“Heterocyclyl” refers to a stable 3- to 24-membered saturated ring which consists of 2 to 20 carbon atoms and from 1 to 6 heteroatoms selected from atoms consisting of nitrogen, oxygen, or sulfur. Unless stated otherwise specifically in the specification, the heterocyclyl may be a monocyclic, bicyclic, tricyclic or tetracyclic ring system, which may include fused or bridged ring systems; and the nitrogen, carbon or sulfur atoms in the heterocyclyl may be optionally oxidised; the nitrogen atom may be optionally quaternised. A heterocyclyl may be optionally substituted.

“Heterocyclylalkyl” refers to a functional group of the formula —RₐRₑ where Rₐ is an alkylene as defined above and Rₑ is a heterocyclyl as defined above, and if the heterocyclyl is a nitrogen-containing heterocyclyl, the heterocyclyl may be attached to the alkylene at the nitrogen atom. A heterocyclylalkyl may be optionally substituted.

“Heteroaryl” refers to a 5- to 20-membered aromatic ring which consists of 1 to 17 carbon atoms and from 1 to 3 heteroatoms selected from atoms consisting of nitrogen, oxygen and sulfur. The heteroaryl may be a monocyclic, bicyclic, tricyclic or tetracyclic ring system, which may include fused or bridged ring systems. A heteroaryl may be optionally substituted.

“Heteroarylalkyl” refers to a functional group of the formula —RₐR_{f} where Rₐ is an alkylene as defined above and R_{f} is a heteroaryl as defined above. A heteroarylalkyl may be optionally substituted.

“Optionally substituted” means that the subsequently described event of circumstances may or may not occur and that the description includes instances where the event or circumstance occurs and instances in which it does not. For example, unless specified otherwise, “optionally substituted” means that the chemical moiety may or may not be substituted by one or more of the following groups: alkyl, alkenyl, halo, haloalkenyl, cyano, nitro, aryl, cycloalkyl, heterocyclyl, heteroaryl, oxo, —OR¹⁰, —OC(O)—R¹⁰, —N(R¹⁰)₂, —C(O)R¹⁰, —C(O)OR¹⁰, —C(O)N(R¹⁰)₂, —N(R¹⁰)C(O)OR¹², —N(R¹⁰)C(O)R¹², —N(R¹⁰)S(O)ₜR¹² (where t is 1 to 2), —S(O)ₜOR¹² (where t is 1 to 2), —S(O)ₓR¹² (where x is 0 to 2) and —S(O)ₜN(R¹⁰)₂ (where t is 1 to 2) where each R¹⁰ is independently hydrogen, alkyl, haloalkyl, cycloalkyl, cycloalkylalkyl, aryl (optionally substituted with one or more halogen groups), aralkyl, heterocyclyl, heterocyclylalkyl, heteroaryl or heteroarylalkyl; and each R¹² is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted unless otherwise indicated.

It is understood that the test methods that are disclosed in the Test Methods section of the present application must be used to determine the respective values of the parameters of the present invention as described and claimed herein.

### Ionic Liquid Sytems

Surprisingly, it has been found that ionic liquids can be used in ionic liquid systems, such that when formulated into consumer products, they can deliver and/or deposit fragrance benefit on a substrate. It has also been discovered that ionic liquid systems comprising at least one ionic liquid can also aid in biofilm removal, freshness delivery, and/or malodour control and/or elimination. In particular, the applicants have discovered that certain perfume raw materials and the correspondent anion can be derived such that the anion can ion pair with a cation and form an ionic liquid. The resulting ionic liquid will deposit on a substrate and release the perfume material upon drying out. For example, with a consumer product that involves machine-washing (e.g., detergents), the ionic liquids can aid in the deposition of the PRMs onto the fabric. This occurs because the anion once deposited on the substrate releases the PRMs upon the fabric drying out. Without wishing to be bound by theory, it is believed that the ionic liquids will form a coacervate with other ingredients in the formula.

With conventional ionic liquids, there is a cation and an anion which are synthesised and then added to an existing composition that contains a fragrance component. The ionic liquids interact (attract/repel) with the PRMs according to electrostatic/hydrogen-bonding non-covalent forces. However, PRMs having low c Log P, preferably below 3, tend to be very hydrophilic and can be difficult to deposit on surfaces, preferably soft surfaces (e.g., fabric, carpet, skin, hair, etc.), as they are typically lost in the through-the-wash process. One solution has been to encapsulate the PRMs in PMC to improve delivery/deposition. Unfortunately, these hydrophilic PRMs tend to be very hard to encapsulate with PMC and even if successful with the encapsulation they tend to leak out of the PMC during storage resulting in, potentially significant, losses over-time.

Accordingly, the ionic liquid system according to the present invention comprises one or more ionic liquids, each comprising a cation and an anion, wherein the conjugate acid of at least one of the anions is a perfume raw material with a pKₐ from about 0 to about 14, preferably from about 0 to about 9, or more preferably from about 4 to about 8.

In an embodiment of the present invention, wherein each ionic liquid comprises a cation independently selected from the group consisting of:

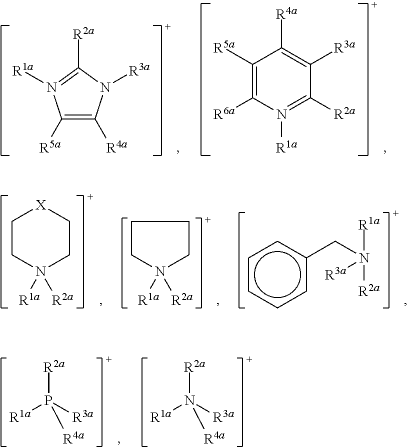

and
combinations thereof;
   wherein: X is CH₂ or O;
each R^{1a}, R^{3a}, and R^{4a} are independently selected from hydrogen, C₁-C₂₀ alkyl, C₁-C₂₀ alkenyl, C₁-C₂₀ alkynyl, C₁-C₂₀ alkoxy, C₁-C₂₀ alkoxyC₁-C₂₀alkyl, C₃-C₇ cycloalkyl, C₃-C₇ cycloalkylC₁-C₄alkyl, C₂-C₂₀ heterocyclyl, C₆-C₁₀ aryl, C₆-C₁₀ arylC₁-C₁₀alkyl, C₁-C₁₀ heteroaryl, halo, haloC₁-C₂₀alkyl, hydroxyl, hydroxyC₁-C₂₀alkyl, or —N(R^{6a})₂;
each R^{2a} is independently selected from hydrogen, C₁-C₂₀ alkyl, C₁-C₂₀ alkenyl, or C₁-C₂₀ alkynyl;
each R^{5a} is independently selected from hydrogen, C₁-C₂₀ alkyl, C₁-C₂₀ alkenyl, C₁-C₂₀ alkynyl, —R^{7a}—OR^{8a}, or —R^{7a}—OR^{7a}—OR^{8a}
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, alkylene chain, alkenylene chain, or alkynylene chain; and
each R^{8a} is independently selected from a hydrogen, alkylene chain, alkenylene chain, or alkynylene chain.

Of this embodiment of the invention, wherein the cation is independently selected from the group consisting of 1-butyl-3-methylimidazolium; (N-ethyl-2-(2-methoxyethoxy)-N,N-dimethylethanaminium); 2-(2-ethoxyethoxy)-N-ethyl-N,N-dimethylethanaminium; N-benzyl-N,N-dimethyloctan-1-aminium; N-benzyl-N,N-dimethylnonan-1-aminium; and combinations thereof.

The methods for preparing the cations of the present invention are provided in the Examples section. The preparations are not intended to limit the scope of the present invention.

In an embodiment of the present invention, wherein the conjugate acid of at least one of the anions is a perfume raw material having a c Log P value between about 0 to about 7, preferably between about 1 to about 3.

In an embodiment of the present invention, wherein the conjugate acid of at least one of the anions is a PRM selected from materials listed in Table 1 hereinafter.

TABLE 1
Ionizable Perfume Raw Materials based on
pKₐ to be used as Anions in Ionic Liquids

| | CAS | | | |
|---|---|---|---|---|
| No. | Number | IUPAC Name | Commercial Name | pKa |
| 1. | 65-85-0 | Benzoic acid | Dracylic acid | 4.1 |
| 2. | 103-82-2 | Benzeneacetic acid | Phenylacetic acid | 4.3 |
| 3. | 100-09-4 | 4-Methoxybenzoic acid | Draconic acid | 4.4 |
| | | | NSC 32742 | |
| | | | NSC 7926 | |
| | | | p-Methoxybenzoic acid | |
| 4. | 140-10-3 | 2-Propenoic acid, 3-phenyl-, | Trans-cinnamic acid | 4.4 |
| | | (2E)- | | |
| 5. | 3142-72-1 | 2-Methyl-2-pentenoic acid | Strawberry acid | 4.6 |
| 6. | 501-52-0 | Benzenepropanoic acid | Hydrocinnamic acid | 4.6 |
| 7. | 334-48-5 | Decanoic acid | Capric acid | 4.9 |
| 8. | 124-07-2 | Octanoic acid | caprylic acid | 4.9 |
| 9. | 143-07-7 | Dodecanoic acid | Lauric acid | 4.9 |
| 10. | 85392-03-6 | 5-Decenoic acid | milk lactone | 4.9 |
| | | | Dec-5-enoic acid | |
| | | | 5-Decylenic acid | |
| 11. | 121-32-4 | 3-Ethoxy-4- | Ethyl vanillin | 7.3 |
| | | hydroxybenzaldehyde | 3-Ethylvanillin | |
| | | | Bourbonal | |
| 12. | 121-33-5 | 4-Hydroxy-3- | Vanillin | 7.3 |
| | | methoxybenzaldehyde | Vanillaldehyde | |
| | | | p-Vanillin | |
| 13. | 93-35-6 | 7-Hydroxy-2H-1-benzopyran-2- | Umbelliferone | 7.8 |
| | | one | Coumarin, 7-hydroxy- | |
| | | | (7CI,8CI) | |
| 14. | 1009-11-6 | 1-(4-Hydroxyphenyl) butan-1- | p-butyrylphenol | 8.1 |
| | | one | Butyrophenone, 4′- | |
| | | | hydroxy- (6CI, 7CI, 8CI) | |
| | | | p-Butyrylphenol | |
| | | | p-Hydroxybutyrophenone | |
| 15. | 121-34-6 | 4-Hydroxy-3-methoxybenzoic | Vanillic acid | 8.5 |
| | | acid | | |
| 16. | 4940-11-8 | 4H-Pyran-4-one, 2-ethyl-3- | Ethyl maltol | 8.7 |
| | | hydroxy- | NSC 638851 | |
| | | | Veltol plus | |
| 17. | 118-71-8 | 3-Hydroxy-2-methyl-4-pyrone | maltol | 8.7 |
| | | | Larixin | |
| | | | NSC 2829 | |
| | | | NSC 404458 | |
| | | | Palatone | |
| | | | Veltol | |
| 18. | 458-36-6 | 3-(4-Hydroxy-3- | 4-hydroxy-3-methoxy- | 9.5 |
| | | methoxyphenyl)prop-2-enal | cinnamaldehyde | |
| | | | Coniferaldehyde | |
| | | | Ferulaldehyde | |
| 19. | 3209-13-0 | 3-Methoxy-5-methylphenol | 5-methoxy-m-Cresol | 9.6 |
| | | | Orsin monomethyl ether | |
| 20. | 88-04-0 | 4-Chloro-3,5-dimethylphenol | Benzytol | 9.6 |
| | | | Finecide C 2000 | |
| | | | Hokubarine E 400 | |
| | | | NSC 4971 | |
| | | | Nipacide PX | |
| | | | Ottasept | |
| | | | Ottasept Extra | |
| | | | PCMX | |
| | | | Parametaxylenol | |
| | | | RBA 777 | |
| | | | p-Chloro-m-Xylenol | |
| 21. | 38462-22-5 | 5-Methyl-2-(2-sulfanylpropan- | thiomenthone | 9.6 |
| | | 2-yl)cyclohexan-1-one | buchu ketone | |
| | | | buchu mercaptan | |
| | | | Jallione | |
| | | | ribes mercaptan | |
| | | | thio menthone natural | |
| 22. | 5471-51-2 | 4-(4-Hydroxyphenyl)butan-2- | Raspberry ketone | 9.7 |
| | | one | | |
| 23. | 122-48-5 | 4-(4-Hydroxy-3- | zingerone | 9.7 |
| | | methoxyphenyl)butan-2-one | gingerone | |
| | | | vanillyl acetone | |
| | | | vanillylacetone | |
| 24. | 131-57-7 | (2-Hydroxy-4-methoxyphenyl)- | advastab 45 | 9.7 |
| | | phenylmethanone | cyasorb UV 9 | |
| | | | eusolex 4360 | |
| | | | neo heliopan BB | |
| | | | presun 15 | |
| | | | syntase 62 | |
| | | | uvinul M-40 | |
| 25. | 2050-08-0 | Pentyl 2-hydroxybenzoate | Amyl salicylate | 9.8 |
| | | | Salicylic acid, amyl ester | |
| | | | Salicylic acid, pentyl ester | |
| | | | NSC 403668 | |
| | | | NSC 44877 | |
| | | | NSC 46125 | |
| | | | Pentyl salicylate | |
| 26. | 2052-14-4 | Butyl 2-hydroxybenzoate | Butyl salicylate | 9.8 |
| | | | NSC 1511 | |
| | | | NSC 403676 | |
| | | | Nefolia | |
| | | | Salicylic acid, butyl ester | |
| 27. | 65405-77-8 | [(Z)-hex-3-enyl] 2- | cis-3-hexenyl salicylate | 9.8 |
| | | hydroxybenzoate | hexenyl-3-cis salicylate | |
| | | | leaf salicylate | |
| 28. | 25485-88-5 | Cyclohexyl 2-hydroxybenzoate | Cyclohexyl salicylate | 9.8 |
| | | | salicylic acid, | |
| | | | cyclohexyl ester | |
| | | | NSC 406675 | |
| 29. | 118-61-6 | Ethyl 2-hydroxybenzoate | Mesotol | 9.8 |
| | | | Sal ether | |
| | | | Salicylic acid ethyl ester | |
| | | | Salicylic ether | |
| | | | Salicylic ethyl ester | |
| | | | NSC 8209 | |
| 30. | 87-20-7 | 3-Methylbutyl 2- | Isoamyl salicylate | 9.8 |
| | | hydroxybenzoate | Iso pentyl salicylate | |
| | | | NSC 7952 | |
| 31. | 87-19-4 | 2-Methylpropyl 2- | Isobutyl salicylate | 9.8 |
| | | hydroxybenzoate | NSC 62140 | |
| | | | salicylic acid isobutyl ester | |
| 32. | 119-36-8 | Methyl 2-hydroxybenzoate | Methyl salicylate | 9.8 |
| | | | Salicylic acid, | |
| | | | methyl ester | |
| | | | Analgit | |
| | | | Anthrapole ND | |
| | | | Flucarmit | |
| | | | NSC 8204 | |
| 33. | 6259-76-3 | Hexyl 2-hydroxybenzoate | N-hexyl salicylate | 9.8 |
| | | | salicylic acid hexyl ester | |
| 34. | 68555-58-8 | 3-Methylbut-2-enyl 2- | Prenyl salicylate | 9.8 |
| | | hydroxybenzoate | 2-iso pentenyl salicylate | |
| 35. | 118-58-1 | Benzyl 2-hydroxybenzoate | Benzyl salicylate | 9.8 |
| | | | Salicylic acid, | |
| | | | benzyl ester | |
| | | | NSC 6647 | |
| 36. | 77-93-0 | Triethyl 2-hydroxypropane- | Triethyl citrate | 9.9 |
| | | 1,2,3-tricarboxylate | Citroflex 2 | |
| | | | Citroflex C 2 | |
| | | | Citroflex EC | |
| | | | Citroflex SC 60 | |
| | | | Morflex C 2 | |
| | | | NSC 8907 | |
| 37. | 105-67-9 | 2,4-Dimethylphenol | 2,4-Xylenol | 10 |
| | | | NSC 3829 | |
| | | | m-Xylenol | |
| | | | Bacticin | |
| | | | Gallex | |
| 38. | 2785-89-9 | 4-Ethyl-2-methoxyphenol | Homo creosol | 10 |
| | | | NSC 82313 | |
| 39. | 2628-17-3 | 4-Ethenylphenol | 4-vinylphenol | 10 |
| | | | 4-hydroxystyrene | |
| | | | p-hydroxystyrene | |
| | | | p-vinyl phenol | |
| 40. | 499-75-2 | 2-Methyl-5-propan-2-ylphenol | Carvacrol | 10 |
| | | | Antioxine | |
| | | | Caswell no. 511 | |
| | | | p-cymene-2-ol | |
| | | | 2-para-cymenol | |
| | | | Cymophenol | |
| | | | Hydroxy-p-cymene | |
| | | | karvakrol | |
| | | | Dentol | |
| | | | Isothymol | |
| | | | NSC 6188 | |
| 41. | 97-54-1 | 2-Methoxy-4-[(Z)-prop-1- | cis-iso-eugenol | 10 |
| | | enyl]phenol | | |
| 42. | 93-51-6 | 2-Methoxy-4-methylphenol | Creosol | 10 |
| | | | Rohkcrsol | |
| | | | Valspice | |
| | | | NSC 4969 | |
| 43. | 2785-87-7 | 2-Methoxy-4-propylphenol | Dihydroeugenol | 10 |
| | | | Cerulignol | |
| | | | Coerulignol | |
| | | | Dihydroeugenol | |
| | | | Guaiacyl propane | |
| 44. | 97-53-0 | 2-Methoxy-4-prop-2- | Eugenol | 10 |
| | | enylphenol | | |
| 45. | 90-05-1 | 2-Methoxyphenol | Guaiacol | 10 |
| | | | Anastil | |
| | | | o-guaiacol | |
| | | | Guaicolina | |
| | | | Guajacol | |
| | | | Guasol | |
| | | | Pyroguaiac acid | |
| | | | Guaiastil | |
| | | | NSC 3815 | |
| 46. | 108-95-2 | Phenol | 2-Allphenol | 10 |
| | | | Benzenol | |
| | | | Carbolic acid | |
| | | | ENT 1814 | |
| | | | Hydroxybenzene | |
| | | | NSC 36808 | |
| | | | Oxybenzene | |
| | | | Phenic acid | |
| | | | Phenyl alcohol | |
| 47. | 97-54-1 | 2-Methoxy-4-prop-1-en-2- | Isoeugenol | 10 |
| | | ylphenol | | |
| 48. | 620-17-7 | 3-Ethylphenol | m-ethylphenol | 10 |
| 49. | 95-48-7 | 2-Methylphenol | o-cresol | 10 |
| | | | NSC 23076 | |
| | | | NSC 36809 | |
| 50. | 106-44-5 | 4-Methylphenol | p-cresol | 10 |
| | | | NSC 3696 | |
| 51. | 123-07-9 | 4-Ethylphenol | p-ethylphenol | 10 |
| | | | NSC 62012 | |
| 52. | 98-54-4 | 4-tert-Butylphenol | p-tert-butylphenol | 10 |
| | | | 4-(1,1- | |
| | | | Dimethylethyl)phenol | |
| | | | Butylphen | |
| | | | NSC 3697 | |
| | | | PTBP | |
| | | | 1-hydroxy-4-tert-butyl | |
| | | | benzene | |
| 53. | 5932-68-3 | 2-Methoxy-4-[(E)-prop-1- | trans-isoeugenol | 10 |
| | | enyl]phenol | NSC 209522 | |
| 54. | 2563-07-7 | 2-Ethoxy-4-methylphenol | Ultravanil | 10 |
| | | | 2-ethoxy-p-Cresol | |
| | | | 4-Methyl-2-ethoxyphenol | |
| | | | ethoxy-p-cresol | |
| | | | Supravanil | |
| | | | Vanilla cresol | |
| 55. | 94-86-0 | 2-Ethoxy-5-prop-1-enylphenol | Vanitrope | 10 |
| | | | Hydroxy methyl anethol | |
| | | | Isosafroeugenol | |
| | | | NSC 5194 | |
| | | | Propenylguaethol | |
| | | | Ethoxypropenyl phenol | |
| 56. | 128489-02-1 | Phenol, 4-(3,6-dihydro-4- | Eugewhite Isomer 1 | 10 |
| | | methyl-2H-pyran-2-yl)-2- | | |
| | | methoxy- | | |
| 57. | 128489-04-3 | Phenol, 2-methoxy-4- | Eugewhite Isomer 2 | 10 |
| | | (tetrahydro-4-methylene-2H- | | |
| | | pyran-2-yl)- | | |
| 58. | 5595-79-9 | 2-Ethoxy-4- | Methyl diantilis | 10.1 |
| | | (methoxymethyl)phenol | dianthus ethone | |
| 59. | 09-03-33 | 2-Methoxy-4- | vaniwhite | 10.1 |
| | | (methoxymethyl)phenol | Methyl vanillyl ether | |
| | | | Vanillyl alcohol methyl | |
| | | | ether | |
| 60. | 4707-47-5 | Methyl 2,4-dihydroxy-3,6- | Veramoss | 10.2 |
| | | dimethylbenzoate | Evemilox | |
| | | | Evemyl | |
| | | | Oakmoss #1 | |
| | | | Phenomoss | |
| | | | Rrionyl | |
| | | | Verymoss | |
| 61. | 141-97-9 | Ethyl 3-oxobutanoate | Ethyl acetoacetate | 10.6 |
| | | | NSC 37390 | |
| | | | NSC 8657 | |
| 62. | 2308-18-1 | 3-Methylbutyl 3-oxobutanoate | Isoamyl acetoacetate | 10.6 |
| 63. | 89-83-8 | 5-Methyl-2-propan-2-ylphenol | Thymol | 10.8 |
| | | | Thyme camphor | |
| | | | Thymic acid | |
| | | | NSC 11215 | |
| | | | NSC 47821 | |
| | | | NSC 49142 | |
| 64. | 128-37-0 | 2,6-Ditert-butyl-4-methylphenol | Antox QT | 12.1 |
| | | | BHT | |
| | | | Di-tert-Butyl-4- | |
| | | | methylphenol | |
| | | | Ionol | |
| | | | Tenox BHT | |
| | | | Tonarol | |
| | | | Benzoin | |
| 65. | 119-53-9 | 2-Hydroxy-1,2- | Benzoylphenylcarbinol | 12.3 |
| | | diphenylethanone | DL-Benzoin | |
| | | | Desyl alcohol | |
| | | | Esacure EB 3 | |
| | | | NSC 8082 | |
| | | | Nisso Cure MB | |
| | | | S 19 | |
| | | | Seikuol Z | |
| | | | Wy 42956 | |
| 66. | 105-53-3 | Diethyl propanedioate | Diethyl malonate | 13 |
| | | | NSC 136903 | |
| | | | NSC 8864 | |
| 67. | 29214-60-6 | Ethyl 2-acetyloctanoate | Gelsone | 13 |
| | | | Jasmine acetoacetate | |
| | | | Jaswamin | |
| | | | Jessate | |
| 68. | 25395-31-7 | (1-Acetyloxy-3-hydroxypropan- | Diacetin | 13.6 |
| | | 2-yl) acetate | Glycerol diacetate | |
| | | | Glyceryl diacetate | |
| 69. | 513-86-0 | 3-Hydroxybutan-2-one | Acetoin | 13.9 |
| | | | NSC 7609 | |

* pKₐ were calculated utilizing the ACD Software V.14.02, as described in the Test Methods Section.
** Origin: The PRMs may be obtained from one or more of the following companies: Firmenich (Geneva, Switzerland), Symrise AG (Holzminden, Germany), Givaudan (Argenteuil, France), IFF (Hazlet, New Jersey), Bedoukian (Danbury, Connecticut), Sigma Aldrich (St. Louis, Missouri), Millennium Speciality Chemicals (Olympia Fields, Illinois), Polarone International (Jersey City, New Jersey), and Aroma & Flavor Specialities (Danbury, Connecticut).

Preferred ionisable perfume raw materials whose anions can be used as anions in ionic liquids of the present invention are those listed in Table 1. Preferably, the ionizable perfume raw material is selected from the group consisting of: benzoic acid; benzeneacetic acid; 4-methoxy benzoic acid; 2-propenoic acid, 3-phenyl-, (2E)-; 2-methyl-2-pentenoic acid; benzenepropanoic acid; decanoic acid; octanoic acid; dodecanoic acid; 5-decenoic acid; 3-ethoxy-4-hydroxybenzaldehyde; 4-hydroxy-3-methoxybenzaldehyde; 7-hydroxy-2H-1-benzopyran-2-one; and combinations thereof, preferably 3-ethoxy-4-hydroxybenzaldehyde; 4-hydroxy-3-methoxybenzaldehyde; and combinations thereof.

However, it is understood by one skilled in the art that other ionizable perfume raw materials, which originates anions, and which are not recited in Table 1, would also fall within the scope of the present invention, so long as they are perfume raw materials having a pKₐ of from about 0 to about 14, preferably from about 0 to 8, or more preferably from about 4 to 8.

Preferably, the ionic liquids useful in the present invention exhibit no measurable vapour pressure between 25° C. and 100° C. Thus, it is understood that the ionic liquids themselves make no contribution to the vapour pressure of any mixture in which they are incorporated.

As used herein, the term “ionic liquid” refers to a liquid which consists excusively of ions and is preferably present in a liquid form at temperatures lower than 100° C., preferably at ambient or room temperature (i.e., from 15° C. to 30° C.). Particularly preferred ionic liquids are suitable for use in consumer products and have to be chosen so as to avoid adverse effect in terms of health and/or the environment.

Ionic liquids have no effective vapour pressure (essentially zero) and may be easy to handle. Their solvent properties can be readily adjusted so as to be suitable to a wide range of PRMs. Solvent properties can be readily adjusted by adjusting the structural features of both cation and anions of ionic liquids. The solvent properties can be systematically altered to suit the purpose. Diverse groups on cation and anion will change dielectric, hydrogen-bond donor and hydrogen-bond acceptor abilities of ionic liquids. As a consequence, their interactions with PRMs will change accordingly.

Typically, ionic liquids may have high viscosities (i.e., greater than about 1,000 mPa·s) at room temperature. High viscosities can be problematic in formulating the compositions of the present invention. Therefore, in an embodiment, the present invention is preferably directed to ionic liquids (undiluted with adjuncts, co-solvents or free water) which have viscosities of less than about 1000 mPa·s, preferably less than about 750 mPa·s, preferably less than about 500 mPa·s, as measured at 20° C. In some embodiments, the viscosity of the undiluted ionic liquids are in the range from about 1 mPa·s to about 400 mPa·s, preferably from 1 mPa·s to about 300 mPa·s, and more preferably from about 1 mPa·s to about 250 mPa·s.

The viscosities of the ionic liquids and compositions containing therein can be measured on a Brookfield viscometer model number LVDVII+ at 20° C., with Spindle S31 at the appropriate speed to measure materials of differing viscosities. Typically, the measurement is performed at a speed from 12 rpm to 60 rpm. The undiluted state is prepared by storing the ionic liquids in a desiccator containing a desiccant (e.g. anhydrous calcium chloride) at room temperature for at least 48 hours prior to the viscosity measurement. This equilibration period unifies the amount of innate water in the undiluted samples.

The ionic liquids may be used in the compositions and/or consumer products of the present invention as pure solvents (i.e., as a pure, undiluted ionic liquid); as a co-solvent in conjunction with water, or organic solvents; or as an active where the continuous phase is water or another solvent. Various adjunct ingredients known in the art may be incorporated into such compositions. In certain embodiments, water and/or solvent may be present in the composition at least about 0.01 wt % or at least about 1 wt % or at least about 10 wt %, and less than about 50 wt % or less than about 30 wt % or less than about 20 wt % by weight of the composition.

It should be understood that the terms “ionic liquid system” refers to a system comprising one or more ionic liquids. In an embodiment, the ionic liquid system comprises two, three, four, five or more ionic liquids. The ionic liquid system may be formed from a homogeneous combination comprising one species of anion and one species of cation, or it can be composed of more than one species of cation and/or anion. Thus, an ionic liquid may be composed of more than one species of cation and one species of anion. An ionic liquid may further be composed of one species of cation and more than one species of anion. Finally, an ionic liquid may further be composed of more than one species of cation and more than one species of anion.

In another embodiment of the present invention, the ionic liquids, preferably the anion component may be selectively made to be hydrophobic.

In yet another embodiment of the present invention, wherein at least one of the ionic liquid comprises anions whose conjugate acids are not perfume raw materials and are independently selected from the group consisting of:

[R¹—O—C(O).CH(SO₃)R³—C(O).O—R²]⁻ (I)

(a)

wherein:
R¹ and R² are independently selected from the group consisting of alkyl or alkenyl, provided that the alkyl is not substituted with nitro, azido or halide; and
R³ is alkylene, heteroarylene, arylene, or cycloalkylene;

(b)

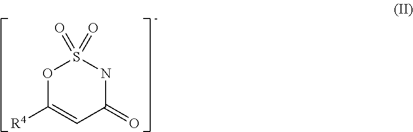

wherein:
R⁴ is selected from the group consisting of hydrogen, cyano, alkyl, alkoxy and alkoxyalkyl;

(c)

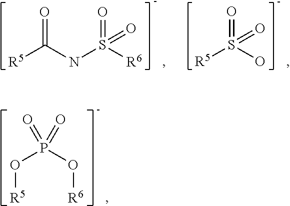

bistriflamide and
combinations thereof;
wherein:
each R⁵ and R⁶ are independently selected from the group consisting of hydrogen, alkyl, alkenyl, alkynyl, alkoxyalkyl, cycloalkyl, cycloalkylalkyl, aryl, aralkyl, heterocyclyl, heterocyclyalkyl, heteroaryl and heteroarylalkyl; and

(d) combinations thereof.

Of this embodiment, wherein the anions whose conjugate acids are not perfume raw materials and are independently selected from the group consisting of: 1,4-bis(2-ethylhexoxy)-1,4-dioxobutane-2-sulfonate; 6-methyl-3,4-dihydro-1,2,3-oxathiazin-4-one 2,2-dioxide; and combinations thereof.

The methods for preparing the anions and the conjugate acids of the present invention are provided in the Examples section. The preparations are not intended to limit the scope of the present invention.

In another embodiment of the present invention, wherein the ionic liquid system further comprises a perfume microcapsule comprising from about 1% w/w to about 90% w/w, based on the total perfume microcapsule weight, of one or more perfume raw materials. Of this embodiment, wherein the perfume raw materials comprise materials selected from the group consisting of:
a) a perfume composition having a C log P of less than 4.5;
b) a perfume composition comprising, based on total perfume composition weight, 60% perfume materials having a C log P of less than 4.0;
c) a perfume composition comprising, based on total perfume composition weight, 35% perfume materials having a C log P of less than 3.5;
d) a perfume composition comprising, based on total perfume composition weight, 40% perfume materials having a C log P of less than 4.0 and at least 1% perfume materials having a C log P of less than 2.0;
e) a perfume composition comprising, based on total perfume composition weight, 40% perfume materials having a C log P of less than 4.0 and at least 15% perfume materials having a C log P of less than 3.0;
f) a perfume composition comprising, based on total perfume composition weight, at least 1% butanoate esters and at least 1% of pentanoate esters;
g) a perfume composition comprising, based on total perfume composition weight, at least 2% of an ester comprising an allyl moiety and at least 10% of another perfume comprising an ester moiety;
h) a perfume composition comprising, based on total perfume composition weight, at least 1% of an aldehyde comprising an alkyl chain moiety;
i) a perfume composition comprising, based on total perfume composition weight, at least 2% of a butanoate ester;
j) a perfume composition comprising, based on total perfume composition weight, at least 1% of a pentanoate ester;
k) a perfume composition comprising, based on total perfume composition weight, at least 3% of an ester comprising an allyl moiety and 1% of an aldehyde comprising gan alkyl chain moiety;
l) a perfume composition comprising, based on total perfume composition weight, at least 25% of a perfume comprising an ester moiety and 1% of an aldehyde comprising an alkyl chain moiety;
m) a perfume composition comprising, based on total perfume composition weight, at least 2% of a material selected from the group consisting of 4-(2,6,6-trimethyl-1-cyclohexenyl)-3-buten-2-one; 4-(2,6,6-trimethyl-2-cyclohexenyl)-3-buten-2-one; 3-buten-2-one,3-methyl-4-(2,6,6-trimehtyl-1-cyclohexen-2-yl)-; and mixtures thereof;
n) a perfume composition comprising, based on total perfume composition weight, at least 0.1% of tridec-2-enonitrile; mandaril; or mixtures thereof;
o) a perfume composition comprising, based on total perfume composition weight, at least 2% of a material selected from 3,7-dimethyl-6-octenenitrile; 2-cyclohexylidene-2-phenylacetonitrile; or mixtures thereof;
p) a perfume composition comprising, based on total perfume composition weight, at least 80% of one or more perfumes comprising a moiety selected from the group consisting of esters, aldehydes, ionones, nitriles, ketones and combinations thereof;
q) a perfume composition comprising, based on total perfume composition weight, at least 3% of an ester comprising an allyl moiety; a perfume composition comprising, based on total perfume composition weight, at least 20% of a material selected from the group consisting of: 1-methylethyl-2-methylbutanoate; ethyl-2-methyl pentanoate; 1,5-dimethyl-1-ethenylhexyl-4-enyl acetate; p-meth-1-en-8-yl acetate; 4-(2,6,6-trimethyl-2-cyclohexenyl)-3-buten-2-one; 4-acetoxy-3-methoxy-1-propenylbenzene; 2-propenyl cyclohexanepropionate; bicyclo[2.2.1]hept-5-ene-2-carboxylic acid,3-(1-methylethyl)-ethyl ester; bycyclo[2.2.1]heptan-2-ol, 1,7,7-trimethyl-, acetate; 1,5-dimethyl-1-ethenylhex-4-enylacetate; hexyl 2-methyl propanoate; ethyl-2-methylbutanoate; 4-undecanone; 5-heptyldihydro-2(3h)-furanone; 1,6-nonadien-3-ol,3,7dimethyl-; 3,7-dimethylocta-1,6-dien-3-o; 3-cyclohexene-1-carboxaldehyde,dimethyl-; 3,7-dimethyl-6-octene nitrile; 4-(2,6,6-trimethyl-1-cyclohexenyl)-3-buten-2-one; tridec-2-enonitrile; patchouli oil; ethyl tricycle [5.2.1.0]decan-2-carboxylate; 2,2-dimethyl-cyclohexanepropanol; hexyl ethanoate, 7-acetyl,1,2,3,4,5,6,7,8-octahydro-1,1,6,7-tetramethyl naphtalene; allyl-cyclohexyloxy acetate; methyl nonyl acetic aldehyde; 1-spiro[4,5]dec-7-en-7-yl-4-pentenen-1-one; 7-octen-2-ol,2-methyl-6-methylene-,dihydro; cyclohexanol,2-(1,1-dimethylethyl)-, acetate; hexahydro-4,7-methanoinden-5(6)-yl propionatehexahydro-4,7-methanoinden-5(6)-yl propionate; 2-methoxynaphtalene; 1-(2,6,6-trimethyl-3-cyclohexenyl)-2-buten-1-one; 1-(2,6,6-trimethyl-2-cyclohexenyl)-2-buten-1-one; 3,7-dimethyloctan-3-ol; 3-buten-2-one,3-methyl-4-(2,6,6-trimehtyl-1-cyclohexen-2-yl)-; hexanoic acid, 2-propenyl ester; (Z)-non-6-en-1-al; 1-decyl aldehyde; 1-octanal; 4-t-butyl-α-methylhydrocinnamaldehyde; α-hexylcinnamaldehyde; ethyl-2,4-hexadienoate; 2-propenyl 3-cyclohexanepropanoate; and mixtures thereof;
r) a perfume composition comprising, based on total perfume composition weight, at least 20% of a material selected from the group consisting of: 1-methylethyl-2-methylbutanoate; ethyl-2-methyl pentanoate; 1,5-dimethyl-1-ethenylhex-4-enyl acetate; p-menth-1-en-8-yl acetate; 4-(2,6,6-trimethyl-2-cyclohexenyl)-3-buten-2-one; 4-acetoxy-3-methoxy-1-propenylbenzene; 2-propenyl cyclohexanepropionate; bicyclo[2.2.1]hept-5-ene-2-carboxylic acid,3-(1-methylethyl)-ethyl ester; bycyclo [2.2.1]heptan-2-ol, 1,7,7-trimethyl-, acetate; 1,5-dimethyl-1-ethenylhex-4-enyl acetate; hexyl 2-methyl propanoate; ethyl-2-methylbutanoate,4-undecanolide; 5-heptyldihydro-2(3h)-furanone; 5-hydroxydodecanoic acid; decalactones; undecalactones, 1,6-nonadien-3-ol,3,7dimethyl-; 3,7-dimethylocta-1,6-dien-3-ol; 3-cyclohexene-1-carboxaldehyde,dimethyl-; 3,7-dimethyl-6-octene nitrile; 4-(2,6,6-trimethyl-1-cyclohexenyl)-3-buten-2-one; tridec-2-enonitrile; patchouli oil; ethyl tricycle [5.2.1.0]decan-2-carboxylate; 2,2-dimethyl-cyclohexanepropanol; allyl-cyclohexyloxy acetate; methyl nonyl acetic aldehyde; 1-spiro[4,5]dec-7-en-7-yl-4-pentenen-1-one; 7-octen-2-ol,2-methyl-6-methylene-,dihydro, cyclohexanol,2-(1,1-dimethylethyl)-, acetate; hexahydro-4,7-methanoinden-5(6)-yl propionatehexahydro-4,7-methanoinden-5(6)-yl propionate; 2-methoxynaphtalene; 1-(2,6,6-trimethyl-3-cyclohexenyl)-2-buten-1-one; 1-(2,6,6-trimethyl-2-cyclohexenyl)-2-buten-1-one; 3,7-dimethyloctan-3-ol; 3-buten-2-one,3-methyl-4-(2,6,6-trimehtyl-1-cyclohexen-2-yl)-; hexanoic acid, 2-propenyl ester; (Z)-non-6-en-1-al; 1-decyl aldehyde; 1-octanal; 4-t-butyl-α-methylhydrocinnamaldehyde; ethyl-2,4-hexadienoate; 2-propenyl 3-cyclohexanepropanoate; and mixtures thereof;
s) a perfume composition comprising, based on total perfume composition weight, at least 5% of a material selected from the group consisting of 3-cyclohexene-1-carboxaldehyde,dimethyl-; 3-buten-2-one,3-methyl-4-(2,6,6-trimethyl-1-cyclohexen-2-yl)-; patchouli oil; Hexanoic acid, 2-propenyl ester; 1-Octanal; 1-decyl aldehyde; (Z)-non-6-en-1-al; methyl nonyl acetic aldehyde; ethyl-2-methylbutanoate; 1-methylethyl-2-methylbutanoate; ethyl-2-methyl pentanoate; 4-hydroxy-3-ethoxybenzaldehyde; 4-hydroxy-3-methoxybenzaldehyde; 3-hydroxy-2-methyl-4-pyrone; 3-hydroxy-2-ethyl-4-pyrone; and mixtures thereof;
t) a perfume composition comprising, based on total perfume composition weight, less than 10% perfume materials having a C log P greater than 5.0;
u) a perfume composition comprising geranyl palmitate;
v) a perfume composition comprising a first and an optional second material, the first material having:
   (i) a C log P of at least 2; and
   (ii) a boiling point of less than about 280° C.;
      wherein, the optional second material, if present, having a C log P of less than 2.5; and
w) combinations thereof.

In yet another embodiment of the present invention, the ionic liquid systems (i.e., cation and anion) are essentially free of any of the following chemical moieties: antimony, barium, beryllium, bromine, cobalt, chromium, iodine, lead, nickel, selenium, or thallium. By “essentially free” it is meant that no cation or anion containing any of the foregoing chemical moieties are intentionally added to form the ionic liquids of the present invention. The term “essentially free” also means that no or negligible levels of impurities or intermediates containing any of the foregoing chemical moieties are formed during the synthesis of the ionic liquids.

It may be advantageous if the composition of the present invention has an ionic liquid which has one or more of the abovementioned salts. It is understood that the ionic liquids can comprise either a single anionic species and a single cationic species or a plurality of different anionic and cationic species. By using different anionic species, the properties of the ionic liquids can be matched in an optimal way to include the desired PRMs and/or other components of the fragrance composition. In an embodiment of the invention, the ionic liquids consist of more than one anionic species.

Ionic liquids are formed by first converting the perfume raw material into a perfume raw material conjugate anion then combining simply salts of a cation and an anion (e.g. sodium salt of the anion and chloride salt of the cation). Ionic liquids lend themselves to preparation via combinatorial chemistry. Some methods for preparing the ionic liquids of the present invention are provided in the Examples section. The preparations are not intended to limit the scope of the present invention.

The present invention also provides an ionic liquid system comprising Σ(I_{N}), wherein “I” represents an ionic liquid and “N” represents an integer from one to fifty, preferably two, three, four, five or more, wherein the ionic liquid system comprises from about 0.1% w/w to about 100% w/w, based on the total system weight, of at least one ionic liquid comprising a cation and an anion, wherein the conjugate acid of the anion is a perfume raw material with a pKₐ from about 0 to about 14, preferably from about 0 to about 8, or more preferably from about 4 to about 8; and wherein the ionic liquid system comprises from about 0% w/w to about 99.9% w/w, based on the total system weight, of at least one ionic liquid comprising anions whose conjugate acids are not perfume raw materials.

### Consumer Products

Applicants have surprisingly found that ionic liquid systems can be incorporated into consumer products to enhance the delivery and/or deposition of a desired scent to such substrate that is contacted with such a product and/or mask an undesirable odour. While current perfume delivery systems (e.g., PMC) provide suitable deposition of desirable odours, they are limited when it comes to scents comprising certain PRMs, preferably having pKₐ from about 7 to about 14 and with a c log P below 3, by which they tend to be hydrophilic in nature. Accordingly, the pool of perfume raw materials available for use in current perfume delivery systems is still limited to meet different scent desires. Thus, the current invention allows formulators a larger pool of perfume raw materials from which to choose from.

The precise level of the ionic liquids and/or ionic liquid systems that is employed depends on the type and end use of the consumer product comprising such materials. Specifically, in one aspect, the present invention provides for a consumer product comprising from about 0.0001% w/w to about 100% w/w, preferably from about 0.01% w/w to about 10% w/w, or more preferably from about 0.1% w/w to about 5% w/w, based on the total consumer product weight, of an ionic liquid system according to the present invention.

In an embodiment, the consumer product of the present invention, wherein the consumer product being a composition intended for the treatment of hard surfaces, soft surfaces, skin or hair.

In another embodiment, the consumer product of the present invention, wherein a 10% solution in water of the consumer product has a pH of from about 1 to about 14, preferably a pH of 7 or higher. Techniques for controlling pH include the use of buffers, alkalis, acids, etc., and are well known to those skilled in the art.

Aspects of the invention include the use of the ionic liquid system of the present invention in a detergent composition. In particular, the present invention provides a detergent composition comprising:
(a) from about 0.001% w/w to about 10% w/w, based on the total weight of the detergent composition, of an ionic liquid system made of at least one ionic liquid comprising:
   (i) an anion, wherein the conjugate acid of the anion is a perfume raw material with a pKₐ of from about 0 to about 14, preferably from about 0 to about 8, or more preferably from about 4 to about 8; and
   (ii) a cation selected from the group consisting of:

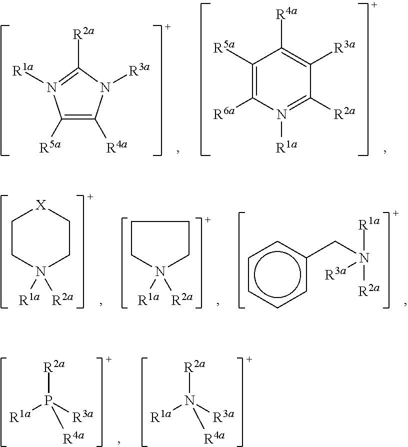

and combinations thereof
X is CH₂ or O;
each R^{1a}, R^{3a}, and R^{4a} are independently selected from hydrogen, C₁-C₂₀ alkyl, C₁-C₂₀ alkenyl, C₁-C₂₀ alkynyl, C₁-C₂₀ alkoxy, C₁-C₂₀ alkoxyC₁-C₂₀alkyl, C₃-C₇ cycloalkyl, C₃-C₇ cycloalkylC₁-C₄alkyl, C₂-C₂₀ heterocyclyl, C₆-C₁₀ aryl, C₆-C₁₀ arylC₁-C₁₀alkyl, C₁-C₁₀ heteroaryl, halo, haloC₁-C₂₀alkyl, hydroxyl, hydroxyC₁-C₂₀alkyl, or —N(R^{6a})_{2;}
each R^{2a} is independently selected from hydrogen, C₁-C₂₀ alkyl, C₁-C₂₀ alkenyl, or C₁-C₂₀ alkynyl;
each R^{5a} is independently selected from hydrogen, C₁-C₂₀ alkyl, C₁-C₂₀ alkenyl, C₁-C₂₀ alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, alkylene chain, alkenylene chain, or alkynylene chain; and
each R^{8a} is independently selected from a hydrogen, alkylene chain, alkenylene chain, or alkynylene chain.

Of this embodiment, the detergent composition further comprising: (b) from about 1% w/w to about 50% w/w, based on the total weight of the detergent composition, of a detersive surfactant.

Of this embodiment, wherein the ionic liquid system is provided in a perfume microcapsule, which optionally further comprises one or more perfume raw materials.

In one aspect, the microcapsule wall material may comprise: melamine, polyacrylamide, silicones, silica, polystyrene, polyurea, polyurethanes, polyacrylate based materials, polyacrylate esters based materials, gelatin, styrene malic anhydride, polyamides, aromatic alcohols, polyvinyl alcohol and mixtures thereof. In one aspect, said melamine wall material may comprise melamine crosslinked with formaldehyde, melamine-dimethoxyethanol crosslinked with formaldehyde, and mixtures thereof. In one aspect, said polystyrene wall material may comprise polyestyrene cross-linked with divinylbenzene. In one aspect, said polyurea wall material may comprise urea crosslinked with formaldehyde, urea crosslinked with gluteraldehyde, and mixtures thereof. In one aspect, said polyacrylate based wall materials may comprise polyacrylate formed from methylmethacrylate/dimethylaminomethyl methacrylate, polyacrylate formed from amine acrylate and/or methacrylate and strong acid, polyacrylate formed from carboxylic acid acrylate and/or methacrylate monomer and strong base, polyacrylate formed from an amine acrylate and/or methacrylate monomer and a carboxylic acid acrylate and/or carboxylic acid methacrylate monomer, and mixtures thereof.

In one aspect, said polyacrylate ester based wall materials may comprise polyacrylate esters formed by alkyl and/or glycidyl esters of acrylic acid and/or methacrylic acid, acrylic acid esters and/or methacrylic acid esters which carry hydroxyl and/or carboxy groups, and allylgluconamide, and mixtures thereof.

In one aspect, said aromatic alcohol based wall material may comprise aryloxyalkanols, arylalkanols and oligoalkanolarylethers. It may also comprise aromatic compounds with at least one free hydroxyl-group, especially preferred at least two free hydroxy groups that are directly aromatically coupled, wherein it is especially preferred if at least two free hydroxy-groups are coupled directly to an aromatic ring, and more especially preferred, positioned relative to each other in meta position. It is preferred that the aromatic alcohols are selected from phenols, cresoles (o-, m-, and p-cresol), naphthols (alpha and beta-naphthol) and thymol, as well as ethylphenols, propylphenols, fluorphenols and methoxyphenols.

In one aspect, said polyurea based wall material may comprise a polyisocyanate. In some embodiments, the polyisocyanate is an aromatic polyisocyanate containing a phenyl, a toluoyl, a xylyl, a naphthyl or a diphenyl moiety (e.g., a polyisocyanurate of toluene diisocyanate, a trimethylol propane-adduct of toluene diisocyanate or a trimethylol propane-adduct of xylylene diisocyanate), an aliphatic polyisocyanate (e.g., a trimer of hexamethylene diisocyanate, a trimer of isophorone diisocyanate and a biuret of hexamethylene diisocyanate), or a mixture thereof (e.g., a mixture of a biuret of hexamethylene diisocyanate and a trimethylol propane-adduct of xylylene diisocyanate). In still other embodiments, the polyisocyante may be cross-linked, the cross-linking agent being a polyamine (e.g., diethylenetriamine, bis(3-aminopropyl)amine, bis(hexanethylene)triamine, tris(2-aminoethyl)amine, triethylenetetramine, N,N′-bis(3-aminopropyl)-1,3-propanediamine, tetraethylenepentamine, pentaethylenehexamine, branched polyethylenimine, chitosan, nisin, gelatin, 1,3-diaminoguanidine monohydrochloride, 1,1-dimethylbiguanide hydrochloride, or guanidine carbonate).

In one aspect, said polyvinyl alcohol based wall material may comprise a crosslinked, hydrophobically modified polyvinyl alcohol, which comprises a crosslinking agent comprising i) a first dextran aldehyde having a molecular weight of from 2,000 to 50,000 Da; and ii) a second dextran aldehyde having a molecular weight of from greater than 50,000 to 2,000,000 Da.

In one aspect, the perfume microcapsule may be coated with a deposition aid, a cationic polymer, a non-ionic polymer, an anionic polymer, or mixtures thereof. Suitable polymers may be selected from the group consisting of: polyvinylformaldehyde, partially hydroxylated polyvinylformaldehyde, polyvinylamine, polyethyleneimine, ethoxylated polyethyleneimine, polyvinylalcohol, polyacrylates, and combinations thereof. Suitable deposition aids are described herein or well-known to those skilled in the art.

In one aspect, the microcapsule may be a perfume microcapsule. In one aspect, one or more types of microcapsules, for examples two microcapsules types, wherein one of the first or second microcapsules (a) has a wall made of a different wall material than the other; (b) has a wall that includes a different amount of wall material or monomer than the other; or (c) contains a different amount perfume oil ingredient than the other; or (d) contains a different perfnme oil, may be used.

In another embodiment, wherein the detergent composition is substantially free of anti-microbes and anti-effectives and retards bacterial growth upon soaking in a wash liquor thereof with a fabric contaminated with bacteria for 5, 8, 10, 12 or 24 hours at 25° C. versus a control composition lacking an ionic liquid.

### Consumer Product Adjunct Materials

For the purposes of the present invention, the non-limiting list of adjuncts illustrated hereinafter are suitable for use in the instant compositions and may be desirably incorporated in certain embodiments of the invention, for example to assist or enhance performance, for treatment of the substrate to be cleaned or to modify the aesthetics of the composition as is the case with perfumes, colorants, dyes or the like. It is understood that such adjuncts are in addition to the components that are supplied via Applicants' perfume systems. The precise nature of these additional components, and levels of incorporation thereof will depend on the physical form of the composition and the nature of the operation for which it is to be used. Suitable adjunct materials include, but are not limited to, surfactants, builders, chelating agents, dye transfer inhibiting agents, dispersants, enzymes, and enzyme stabilizers, catalytic materials, bleach activators, polymeric dispersing agents, clay soil remove/anti-redeposition agents, brighteners, suds suppressors, dyes, additional perfume and perfume delivery systems, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282; 6,306,812B1 and 6,326,348B1 that are incorporated by reference.

Each adjunct ingredients is not not essential to Applicants' compositions. Thus, certain embodiments of Applicants' compositions do not contain one or more of the adjunct ingredients.

Surfactants—The compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants. Anionic and nonionic surfactants are typically employed if the fabric care product is a laundry detergent. On the other hand, cationic surfactants are typically employed if the fabric care product is a fabric softener. In one embodiment, the non-ionic surfactant may comprise an ethoxylated non-ionic surfactant. Suitable for use herein are the ethoxylated alcohols and ethoxylated alkyl phenols of the formula R(OC₂H₄)n OH, wherein R is selected from the group consisting of aliphatic hydrocarbon radicals containing from about 8 to about 20 carbon atoms and alkyl phenyl radicals in which the alkyl groups contain from about 8 to about 12 carbon atoms, and the average value of n is from about 5 to about 15. The surfactant is typically present at a level of from about 0.1 wt %, from about 1 wt %, or even from about 5 wt %, to about 99.9 wt %, to about 80 wt %, to about 35 wt %, or even to about 30 wt %, based on the total weight of the composition.

Builders—The compositions may also contain from about 0.1% to 80% by weight of a builder. Compositions in liquid form generally contain from about 1% to 10% by weight of the builder component. Compositions in granular form generally contain from about 1% to 50% by weight of the builder component. Detergent builders are well known in the art and can contain, for example, phosphate salts as well as various organic and inorganic nonphosphorus builders. Water-soluble, nonphosphorus organic builders useful herein include the various alkali metal, ammonium and substituted ammonium polyacetates, carboxylates, polycarboxylates and polyhydroxy sulfonates. Examples of polyacetate and polycarboxylate builders are the sodium, potassium, lithium, ammonium and substituted ammonium salts of ethylene diamine tetraacetic acid, nitrilotriacetic acid, oxydisuccinic acid, mellitic acid, benzene polycarboxylic acids, and citric acid. Other polycarboxylate builders are the oxydisuccinates and the ether carboxylate builder compositions comprising a combination of tartrate monosuccinate and tartrate disuccinate. Builders for use in liquid detergents include citric acid. Suitable nonphosphorus, inorganic builders include the silicates, aluminosilicates, borates and carbonates, such as sodium and potassium carbonate, bicarbonate, sesquicarbonate, tetraborate decahydrate, and silicates having a weight ratio of SiO₂ to alkali metal oxide of from about 0.5 to about 4.0, or from about 1.0 to about 2.4. Also useful are aluminosilicates including zeolites.

Chelating Agents—The compositions herein may also optionally contain one or more copper, iron and/or manganese chelating agents. If utilized, chelating agents will generally comprise from about 0.1 wt % by weight of the compositions herein to about 15 wt %, or even from about 3 wt % to about 15 wt % by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.000 wtl %, from about 0.01 wt %, from about 0.05 wt % by weight of the compositions to about 10 wt %, about 2 wt %, or even about 1 wt % by weight of the compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions may contain one or more detergent enzymes which provide cleaning performance and/or fabric care benefits. Examples of suitable enzymes include hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination may be a cocktail of conventional applicable enzymes like protease, lipase, cutinase and/or cellulase in conjunction with amylase. Enzymes can be used at their art-taught levels, for example at levels recommended by suppliers such as Novozymes and Genencor. Typical levels in the compositions are from about 0.0001% to about 5%. When enzymes are present, they can be used at very low levels, e.g., from about 0.001% or lower; or they can be used in heavier-duty laundry detergent formulations at higher levels, e.g., about 0.1% and higher. In accordance with a preference of some consumers for “non-biological” detergents, the compositions may be either or both enzyme-containing and enzyme-free.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methyl-enephosphonic acid) and watersoluble salts thereof. Such catalysts are disclosed in U.S. Pat. No. 4,430,243.

Additional Perfume: The additional perfume component may comprise a component selected from the group consisting of
(1) a perfume microcapsule, or a moisture-activated perfume microcapsule, comprising a perfume carrier and an encapsulated perfume composition, wherein said perfume carrier may be selected from the group consisting of cyclodextrins, starch microcapsules, porous carrier microcapsules, and mixtures thereof; and wherein said encapsulated perfume composition may comprise low volatile perfume ingredients, high volatile perfume ingredients, and mixtures thereof;
(2) a pro-perfume;
(3) a low odour detection threshold perfume ingredients, wherein said low odour detection threshold perfume ingredients may comprise less than about 25%, by weight of the total neat perfume composition; and
(4) mixtures thereof.

Porous Carrier Microcapsule—A portion of the additional perfume can also be absorbed onto and/or into a porous carrier, such as zeolites or clays, to form perfume porous carrier microcapsules in order to reduce the amount of free perfume in the multiple use fabric conditioning composition.

Pro-perfume—The additional perfume may additionally include a pro-perfume. Pro-perfumes may comprise nonvolatile materials that release or convert to a perfume material as a result of, e.g., simple hydrolysis, or may be pH-change-triggered pro-perfumes (e.g. triggered by a pH drop) or may be enzymatically releasable pro-perfumes, or light-triggered pro-perfumes. The pro-perfumes may exhibit varying release rates depending upon the pro-perfume chosen.

The compositions of the present invention can be formulated into any suitable form and prepared by any process well known to those skilled in the art.

### Process of Making Consumer Product

In one aspect, the compositions containing the ionic liquid system disclosed herein can be made by the following process whereby: (i) form the anion by deprotonation of the perfume raw material; (ii) combining the cation and anion as disclosed herein above to form the ionic liquid system, and (iii) adding the ionic liquid system to the consumer product adjunct materials to form the consumer product. In another aspect, after forming the anion per point (i) the cation and anion can be combined directly to form the ionic liquid system and then combined with the consumer product adjunct materials to form the consumer product.

Without wishing to be bound by theory, it is believed that the cation-halide pair can be added directly to the consumer product formulation and the ion-exchange reaction happens in-situ when it is contacted with the PRMs at the appropriate product pH to ionize the perfume raw material and turn it into an anion. As a result, the ionic liquid technology is able to deposit very volatile PRM anions onto a substrate and slowly release the conjugated acid perfume raw material upon drying out.

The ionic liquid system may be combined with such one or more consumer product adjunct materials in one or more forms, including a slurry form, neat particle form and spray dried particle form. The ionic liquid system may be combined with such consumer product adjunct materials by methods commonly known to those skilled in the art including mixing and/or spraying.

The compositions of the present invention can be formulated into any suitable form and prepared by any process chosen by the formulator, suitable non-limiting examples of which are described in U.S. Pat. No. 5,879,584.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Ldige GmbH (Paderborn, Germany), Littleford Day, Inc. (Florence, Ky., U.S.A.), Forberg A S (Larvik, Norway), Glatt Ingenieurtechnik GmbH (Weimar, Germany), Niro (Søborg, Denmark), Hosokawa Bepex Corp. (Minneapolis, Minn., U.S.A.), Arde Barinco (New Jersey, U.S.A.).

### Method of Use

Compositions containing the ionic liquid system disclosed herein can be used to deliver and/or deposit scents to a substrate. Typically, at least a portion of the substrate is contacted with an embodiment of the Applicants' composition, in neat form or diluted in a liquor, for example, a wash liquor and then the substrate may be optionally washed and/or rinsed. In one aspect, a substrate is optionally washed and/or rinsed, contacted with a composition comprising the ionic liquid system according to the present invention and then optionally washed and/or rinsed.

In another aspect, a method of providing enhanced fragrance onto a fabric comprising the steps of optionally washing and/or rinsing the fabric, contacting the fabric with a detergent composition according to the present invention, then optionally washing and/or rinsing the fabric. For purposes of the present invention, washing includes but is not limited to, scrubbing and mechanical agitation.

The fabric may comprise any fabric capable of being laundered or treated in normal consumer use conditions. Liquors that may comprise the disclosed compositions may have a pH of from about 3 to about 11.5. Such compositions are typically employed at concentrations of from about 500 ppm to about 15,000 ppm in solution. When the wash solvent is water, the water temperature typically ranges from about 5° C. to about 90° C. and, when the substrate comprises a fabric, the water to fabric ratio is typically from about 1:1 to about 30:1.

Accordingly, the present invention also relates to methods of using the compositions and consumer products for biofilm removal, freshness delivery and/or malodour control.

In other aspect, the present invention also relates to a method of controlling malodour comprising contacting a substrate comprising a malodour with a composition selected from the group consisting of the composition of the consumer product as disclosed herein above, the detergent composition as disclosed herein above, and mixtures thereof.

In other aspect, the compositions of the present invention can be applied to the substrate as a liquid spray, as an aerosol spray or as a pour-on liquid, which can be poured onto the substrate directly or indirectly via a substrate such as a fibrous web substrate (made by woven, non-woven or knitted technologies), a pulp-based substrate (made by air-felt or wet-laid technologies, including paper towels and tissues), a sponge or a foam substrate. Another mode of use would be to incorporate the compositions comprising the ionic liquid into or onto those substrates (e.g., impregnated in a wipe or a mitten), which would alleviate residue problems in those applications where complete dry down is needed.

### Test Methods

It is understood that the test methods that are disclosed in the Test Methods Section of the present application should be used to determine the respective values of the parameters of Applicants' invention as such invention is described and claimed herein.

### Test Method 1: c Log P

The log P values of many perfume ingredients have been reported; for example, the Pomona92 database, available from Daylight Chemical Information Systems, Inc. (Daylight CIS, Irvine, Calif.), contains many, along with citations to the original literature. However, the log P values are most conveniently calculated by the “C LOG P” program, also available from Daylight CIS. This program also lists experimental log P values when they are available in the Pomona92 database. The “calculated log P” (c log P) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990, incorporated herein by reference). The fragment approach is based on the chemical structure of each perfume ingredient, and takes into account the numbers and types of atoms, the atom connectivity, and chemical bonding. The C log P values, which are the most reliable and widely used estimates for this physicochemical property, are preferably used instead of the experimental log P values in the selection of perfume ingredients which are useful in the present invention.

### Test Method 2: Calculated pKₐ

The apparent acid dissociation constant (i.e., pKₐ) for the perfume raw materials is calculated using the pKₐ calculation module of Advanced Chemistry Development (ACD/Labs) Software V14.02 (© 1994-2014 ACD/Labs)). The Ka is defined the equilibrium constant for a dissociation of an acid (HA) to its conjugate base and a hydrogen ion.

### Test Method 3: Olfactory Test

In order to show the effect of the ionic liquid systems on the delivery and/or deposition of the PRMs in a composition of the present invention, test compositions are made, as described in the Example section, and given to panelists to sample. Different product forms comprising of liquid fabric enhancer (“LFE”), unit dose detergent (“SUD”), and/or heavy duty liquid (“HDL”) are made and tested in the wash condition described below. After washing, the headspace measurement for the wet fabric (“WFO”) and dry fabric (“DFO”) are recorded, whereby an increase in headspace vs. a control reference indicates a higher deposition and consequent release of the PRMs.

1. Product Making:

LFE and SUD products are made containing 3 wt % of ionic liquid system added.

2. Load Composition:

Perfume ballast load is 3 Kg and contains:

(i) 600 g polyster;

(ii) 600 g polycotton;

(iii) 600 g muslin (flat) cotton;

(iv) 600 g knitted cotton; and

(v) 600 g terry towels.

Ballast loads are pre-conditioned: 2×70 g Ariel® Sensitive Detergent, 95° C. wash+2× nil powder, short cotton wash @95° C.

After each wash test ballast load is re-washed: 2×70 g Ariel® Sensitive Detergent, 95° C. wash+2× nil powder, short cotton wash @95° C.

For each wash test we add 6 terry tracers (Maes Textile).

Tracers are pre-conditioned: 2×70 g Ariel® Sensitive Detergent, 95° C. wash+2× nil powder, short cotton wash @95° C. Tracers are not re-used.

3. Wash Conditions:

Before test, washing machine is boiled washed (short cotton wash @95° C.).

Test conditions:
(i) Miele Novotronic W526;
(ii) Short cotton cycle wash at 60° C., 1200 rpm spin speed with either Ariel® Sensitive Detergent powder or SUD;
(iii) Put load in washing machine and add powder in the dispenser or the SUD in the washing chamber;
(iv) Optionally, add a dosage of 35 mL LFE in the dispenser;
(v) Run wash cycle; and
(vi) Evaluate WFO and/or after 1 day line dry for the DFO.

4. Performance Evaluation:

Terry tracers are evaluated by panelists and scored on the Primavera Grade (0-100 scale for intensity, where a 10 point difference is consumer noticeable). Panelists are selected from individuals who are either trained to evaluate fragrances according to the scales below or who have considerable experience of fragrance evaluation in the industry (i.e., experts).

### Test Method 4: Anti-Malodour Performance Test

1. Selection and Training of Assessors
A. The assessors must be able to differentiate the sweat odour from an odourless solvent (e.g., dipropylene glycol=DPG). To do this, several cardboard smelling strips are immersed in a highly dilute solution (0.1% in DPG) of the 3-mercapto-3-methyl-hexan-1-ol. In addition, several smelling strips are immersed in DPG. Only those assessors who can perfectly differentiate the smelling strips in a test with 3-mercapto-3-methyl-hexan-1-ol-3-mercapto-3-methyl-hexan-1-ol-DPG and DPG-DPG-3-mercapto-3-methyl-hexan-1-ol take part in further testing.
B. Several concentrations of 3-mercapto-3-methyl-hexan-1-ol are then placed in a container with a volume of 7 L filled with air. The samples are sorted by the assessors according to intensity (i.e. odour strength). The series of concentrations must be correctly recognised and evaluated by the assessors. Assessors who have passed both tests can take part in the raw material test described in further test.

2. Material Test Against Sweat Odour
A. The test materials examined are either individual perfume raw materials (PRMs) or perfume oils. The test materials are evaluated by the selected assessors in a pre-defined gaseous sample with regard to intensity and residual odour strength of the 3-mercapto-3-methyl-hexan-1-ol, a target malodour compound.
B. 1 mu/L of the test material and 5 mu/L of the dilute 3-mercapto-3-methyl-hexan-1-ol (0.1% in DPG) respectively are placed in a container with a volume of 7 L filled with air. The samples are kept at room temperature (e.g., 20° C.) for 15 hrs before evaluation. The individual samples are each evaluated by at least 8 assessors by smelling in comparison with a sample just with 3-mercapto-3-methyl-hexan-1-ol-solution. The mean value is then formed from the at least 8 values obtained for the respective raw material. The intensity of a test material describes the intensity of the test material perceived by odour by trained assessors, irrespective of the quality of the odour as a bad odour or pleasant odour. The stronger a test material smells the higher is the level of the intensity. The intensity is evaluated on a scale of 1 to 9. Level 1 means odourless, 9 means very strong odour detected. The term malodour reduction value describes in the present case the difference in intensity, i.e. the difference between the bad odour (malodour) of the malodour standard mixture and the test mixture.
C. The malodour standard mixture without test material, i.e., 3-mercapto-3-methyl-hexan-1-ol, receives intensity 6. The assessors are selected on the basis of their ability to reproducibly evaluate the strengths of odours. The assessors are trained before the series of tests to recognise the odour of 3-mercapto-3-methyl-hexan-1-ol.

### EXAMPLES

The following examples are provided to further illustrate the present invention and are not to be construed as limitations of the present invention, as many variations of the present invention are possible without departing from its spirit or scope.

The structures of the ionic liquids of the present invention can be characterized by various techniques well-known to the skilled person, including for example: ¹H NMR (nuclear magnetic resonance), ¹³C NMR, Halogen analysis and Elemental analysis.

Nuclear magnetic resonance (“NMR”) is spectroscopic technique well-known to the skilled person and used herein to characterize the ionic liquids prepared herein.

Mass Spectrometry (“MS”) is a spectroscopic technique used herein to quantify the mass to charge ratio of particles or molecules. Two different methods of MS are used: Electron Spray MS (“ES-MS”) and Electron Inionisation MS (“EI-MS”). ES-MS is used for non-volatile materials such as the ionic liquids. EI-MS is used for volatile materials such as the precursor materials.

### Example 1

### Synthesis of Ionic Liquids

The general method for synthesising ionic liquids of the present invention consists of: (i) synthesis of chloride or sulfonate ester precursor; (ii) quaternisation of an amine using a chloroalkane or sulfonyl ester in order to obtain ionic liquid with chloride or sulfonate anion; and (iii) metathesis (i.e., anion exchange) reaction in order to create the target ionic liquid. This is illustrated in Reaction Scheme 1.

### Reaction Scheme 1

### General Synthesis of Ionic Liquids

(i) Precursor synthesis step: R—OH+SOCl₂→R—Cl
   R—OH+R′SO₂Cl→R′SO₃R
(ii) Quaternisation step: R—Cl+Amine→[Cation]Cl
   R′SO₃R+Amine→[Cation][R′SO₃]
(iii) Metathesis Step: [Cation]Cl+M [Anion]→[Cation][Anion]+MCl (M=Na or K)

Ionic liquids are formed by combining salts of a cation and an anion (e.g., sodium or potassium salt of the anion and chloride salt of the cation). Different ionic liquids can be synthesised such that the interactions between the ionic liquids and the solutes (i.e., PRMs) are optimised. Ionic liquids lend themselves to preparation via combinatorial chemistry. The steps shown in the Reaction Scheme 1 are described below in more details.

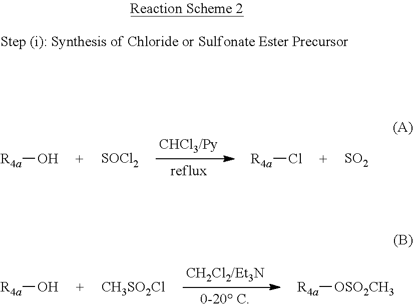

(A) Chloride Precursor Synthesis:

Equimolar amounts of 2-(2-methoxyethoxy)ethanol (1A) or 2-(2-ethoxyethoxy)ethanol (1B) and pyridine are added to a three-neck round bottom flask under N₂. Trichloromethane is used as a solvent for the reaction. Thionyl chloride (1.2 mol eq) is added drop-wise to the stirred mixture via a pressure equalising funnel. Once the addition is completed, the reaction mixture is then heated at 60° C. under reflux for 24 hr. The reaction mixture is then washed with H₂O (4×), saturated aqueous NaHCO₃ (3×), dried over anhydrous MgSO₄ and filtered. The solvent is removed under reduced pressure and the resulting crude product is then distilled yielding the pure product.

(B) Sulfonate Ester Precursor Synthesis:

Equimolar amounts of 2-(2-methoxyethoxy)ethanol (1A) and triethylamine in dichloromethane are added to a round bottom flask in an ice bath under N₂. The mixture is stirred at 0° C. for 20 min before sulfonyl chloride is added drop-wise, in slight excess, via a pressure equalizing funnel. Once the addition is completed, the reaction mixture is warmed to room temperature overnight. The reaction mixture is then washed with H₂O (6×), saturated NaCl solution (3×), dried over anhydrous MgSO₄, filtered and concentrated to yield the sulfonate ester precursor. Sulfonate ester precursor is obtained as a colorless liquid by fractional distillation of the crude product.

TABLE 3
Structures of 1A and 1B

| | Chemical Name | Chemical Structure |
|---|---|---|
| 1A | 2-(2-methoxyethoxy) ethanol | 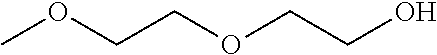 |
| 1B | 2-(2-ethoxyethoxy) ethanol | 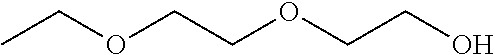 |

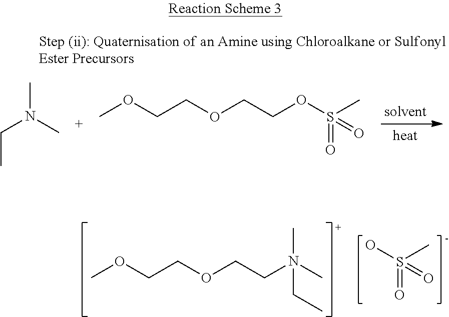

Equimolar amounts of chloride precursor or sulfonyl ester precursor and amine (dimethylethylamine or dimethyloctylamine) are added to a tetrahydrofuran in a sealable reactor. The sealed reaction mixture is stirred and heated at 60° C. until the reaction is completed. The progress of reaction is monitored by NMR spectroscopy. Solvent and unreacted amine are removed under reduced pressure. The product is washed with ethyl ethanoate (6×) and cyclohexane (2×). The residual solvent is removed via rotary evaporator and the product is dried under high vacuum at 40-80° C. for 1-3 days. Exemplary ionic liquids in Table 4 are synthesized according to this method.

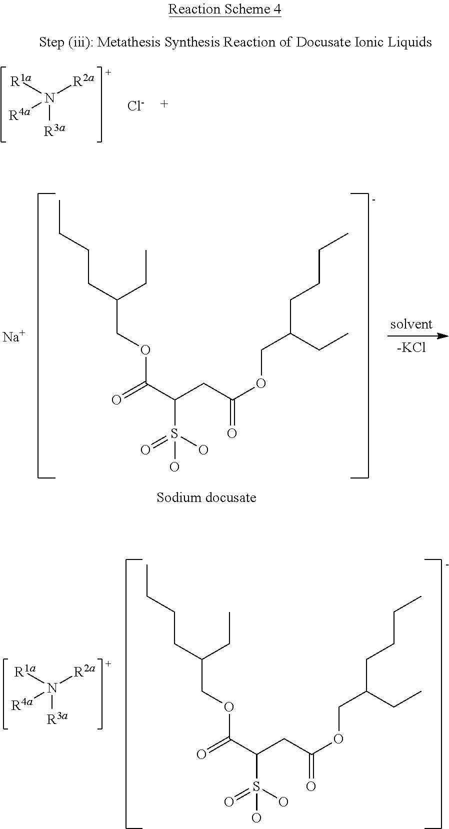

To a chloride ionic liquid in dichloromethane, sodium docusate are added in equimolar quantities, followed by sonication and stirring for 6 hr. The byproduct, sodium chloride, is removed by centrifugation at 4,400 rpm, followed by filtration. The solvent is removed via rotary evaporation. The resulting product is dried by heating at 40-80° C. for 1-3 days, under high vacuum.

TABLE 4
Exemplary Ionic Liquids of the Present Invention

| | | Chemical structure | |
|---|---|---|---|
| Example | IL components | Cation | Anion |
| Ionic Liquid 1 | N-ethyl-2-(2- methoxyethoxy)-N,N- dimethylethananminium 3-ethoxy-4- hydroxybenzaldehyde | | 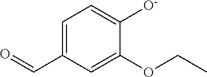 |
| Ionic Liquid 2 | N-benzyl-N,N- dimethylnonan-1- aminium 3-ethoxy-4- hydroxybenzaldehyde | | 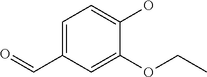 |
| Ionic Liquid 3 | N-ethyl-2-(2- methoxyethoxy)-N,N- dimethylethanaminium 3-ethoxy-4- hydroxybenzaldehyde dioctyl sulfosuccinate | | 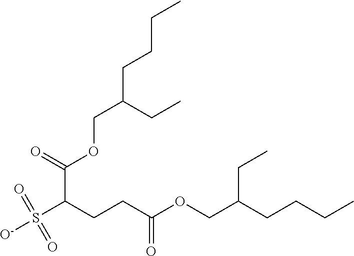 [SMILES: CCCCC(CC)COC(=O)CCC(C(=O)OCC(CC)CCCC)S(=O)(=O)[O-]] |
| Ionic Liquid 4 | N-ethyl-2-(2- methoxyethoxy)-N,N- dimethylethanaminium Hydroxy-3- methoxybenzaldehyde | | 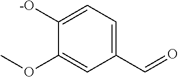 |
| Ionic Liquid 5 | N-benzyl-N,N- dimethylnonan-1- aminium Hydroxy-3- methoxybenzaldehyde | | 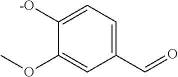 |
| Ionic Liquid 6 | N-ethyl-2-(2- methoxyethoxy)-N,N- dimethylethanaminium Hydroxy-3- methoxybenzaldehyde dioctyl sulfosuccinate | | 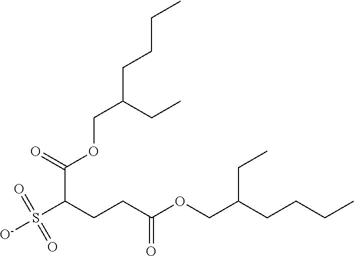 [SMILES: CCCCC(CC)COC(=O)CCC(C(=O)OCC(CC)CCCC)S(=O)(=O)[O-]] |

### Example 2

### Forming the Anion from the Perfume Raw Material

An equimolar mixture of vanillin or ethyl vanillin is treated with sodium methoxide in methanol and stirred for 10 mins. The resulting solution is evaporated on a Rotavap evaporater to remove methanol to obtain sodium vanillinates. Then equimolar mixtures of the appropriate tetraalkylammonium chloride and sodium vanillinate are suspended in dichloromethane and heated for 12 hrs at 40° C. The resulting NaCl byproduct is filtered and the filtrate is evaporated to dryness, kept under high vacuum overnight to obtain the desired ionic liquid. This is illustrated in Reaction Schemes 5a and 5b.

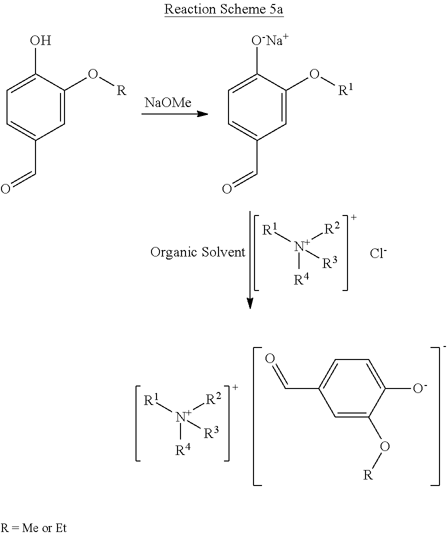

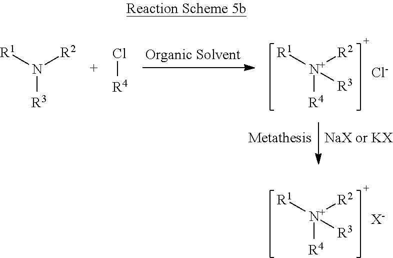

### Example 3

### Detergent Compositions

The following are non-limiting examples of granular detergent compositions containing ionic liquids of the present invention. They are prepared by admixture of the components described in Table 5, in the proportions indicated.

TABLE 5
Granular Detergent Compositions

| | Dry Laundry Compositions (wt %¹) | | | | |
|---|---|---|---|---|---|
| Ingredients | Ex. 1 | Ex. 2 | Ex. 3 | Ex. 4 | Ex. 5 |
| Linear | 20 | 22 | 20 | 15 | 19.5 |
| alkylbenzenesulfonate | | | | | |
| C₁₂₋₁₄ Dimethylhydroxy | 0.7 | 0.2 | 1 | 0.6 | 0.0 |
| ethylammonium chloride | | | | | |
| AE3S | 0.9 | 1 | 0.9 | 0.0 | 0.4 |
| AE7 | 0.0 | 0.0 | 0.0 | 1 | 0.1 |
| Sodium tripolyphosphate | 5 | 0.0 | 4 | 9 | 2 |
| Zeolite A | 0.0 | 1 | 0.0 | 1 | 4 |
| 1.6R Silicate (SiO₂:Na₂O | 7 | 5 | 2 | 3 | 3 |
| at ratio 1.6:1) | | | | | |
| Sodium carbonate | 25 | 20 | 25 | 17 | 18 |
| Polyacrylate MW 4500 | 1 | 0.6 | 1 | 1 | 1.5 |
| Random graft copolymer² | 0.1 | 0.2 | 0.0 | 0.0 | 0.05 |
| Carboxymethyl cellulose | 1 | 0.3 | 1 | 1 | 1 |
| Stainzyme ® (20 mg | 0.1 | 0.2 | 0.1 | 0.2 | 0.1 |
| active/g) | | | | | |
| Protease (Savinase ®, 32.89 mg | 0.1 | 0.1 | 0.1 | 0.1 | 0.1 |
| active/g) | | | | | |
| Amylase - Natalase ® (8.65 mg | 0.1 | 0.0 | 0.1 | 0.0 | 0.1 |
| active/g) | | | | | |
| Lipase - Lipex ® (18 mg | 0.03 | 0.07 | 0.3 | 0.1 | 0.07 |
| active/g) | | | | | |
| Fluorescent Brightener 1 | 0.06 | 0.0 | 0.06 | 0.18 | 0.06 |
| Fluorescent Brightener 2 | 0.1 | 0.06 | 0.1 | 0.0 | 0.1 |
| DTPA | 0.6 | 0.8 | 0.6 | 0.25 | 0.6 |
| MgSO₄ | 1 | 1 | 1 | 0.5 | 1 |
| Sodium Percarbonate | 0.0 | 5.2 | 0.1 | 0.0 | 0.0 |
| Sodium Perborate | 4.4 | 0.0 | 3.85 | 2.09 | 0.78 |
| Monohydrate | | | | | |
| NOBS | 1.9 | 0.0 | 1.66 | 0.0 | 0.33 |
| TAED | 0.58 | 1.2 | 0.51 | 0.0 | 0.015 |
| Sulphonated zinc | 0.0030 | 0.0 | 0.0012 | 0.0030 | 0.0021 |
| phthalocyanine | | | | | |
| S-ACMC | 0.1 | 0.0 | 0.0 | 0.0 | 0.06 |
| Direct Violet Dye (DV9 | 0.0 | 0.0 | 0.0003 | 0.0001 | 0.0001 |
| or DV99 or DV66) | | | | | |
| Neat Perfume² | 0.5 | 0.5 | 0.5 | 0.5 | 0.5 |
| Ionic liquid System from | 3 | 2 | 1 | 0.5 | 1 |
| IL Examples 1 to 6 | | | | | |
| Total | 100 | 100 | 100 | 100 | 100 |

¹wt % relative to the total weight of the composition.
²Optional.

The following are non-limiting examples of liquid detergent compositions containing ionic liquids of the present invention. They are prepared by admixture of the components described in Table 6, in the proportions indicated.

TABLE 6
Liquid Detergent Compositions

| | Liquid Detergent Compositions (wt %¹) | | | | |
|---|---|---|---|---|---|
| Ingredients | Ex. 6 | Ex. 7 | Ex. 8 | Ex. 9 | Ex. 10 |
| AES C₁₂₋₁₅ alkyl ethoxy (1.8) | 11 | 10 | 4 | 6.32 | 0 |
| sulfate | | | | | |
| AE3S | 0 | 0 | 0 | 0 | 2.4 |
| Linear alkyl benzene | 1.4 | 4 | 8 | 3.3 | 5 |
| sulfonate/sulfonic acid | | | | | |
| HSAS | 3 | 5.1 | 3 | 0 | 0 |
| Sodium formate | 1.6 | 0.09 | 1.2 | 0.04 | 1.6 |
| Sodium hydroxide | 2.3 | 3.8 | 1.7 | 1.9 | 1.7 |
| Monoethanolamine | 1.4 | 1.49 | 1.0 | 0.7 | 0 |
| Diethylene glycol | 5.5 | 0.0 | 4.1 | 0.0 | 0 |
| AE9 | 0.4 | 0.6 | 0.3 | 0.3 | 0 |
| AE8 | 0 | 0 | 0 | 0 | 0 |
| AE7 | 0 | 0 | 0 | 0 | 2.4 |
| Chelant (HEDP) | 0.15 | 0.15 | 0.11 | 0.07 | 0.5 |
| Citric Acid | 2.5 | 3.96 | 1.88 | 1.98 | 0.9 |
| C₁₂₋₁₄ dimethyl Amine Oxide | 0.3 | 0.73 | 0.23 | 0.37 | 0 |
| C₁₂₋₁₈ Fatty Acid | 0.8 | 1.9 | 0.6 | 0.99 | 1.2 |
| 4-formyl-phenylboronic acid | 0 | 0 | 0 | 0 | 0.05 |
| Borax | 1.43 | 1.5 | 1.1 | 0.75 | 0 |
| Ethanol | 1.54 | 1.77 | 1.15 | 0.89 | 0 |
| A compound having the | 0.1 | 0 | 0 | 0 | 0 |
| following general structure: | | | | | |
| bis((C₂H₅O)(C₂H₄O)n)(CH₃)—N⁺—CₓH₂ₓ—N⁺—(CH₃)- | | | | | |
| bis((C₂H₅O)(C₂H₄O)n), | | | | | |
| wherein n = from 20 to 30, | | | | | |
| and x = from 3 to 8, or | | | | | |
| sulphated or sulphonated | | | | | |
| variants thereof | | | | | |
| Ethoxylated (EO₁₅) | 0.3 | 0.33 | 0.23 | 0.17 | 0.0 |
| tetraethylene pentamine | | | | | |
| Ethoxylated | 0 | 0 | 0 | 0 | 0 |
| Polyethylenimine | | | | | |
| Ethoxylated hexamethylene | 0.8 | 0.81 | 0.6 | 0.4 | 1 |
| diamine | | | | | |
| 1,2-Propanediol | 0.0 | 6.6 | 0.0 | 3.3 | 0.5 |
| Fluorescent Brightener | 0.2 | 0.1 | 0.05 | 0.3 | 0.15 |
| Hydrogenated castor oil | 0.1 | 0 | 0 | 0 | 0 |
| derivative structurant | | | | | |
| Perfume | 1.6 | 1.1 | 1.0 | 0.8 | 0.9 |
| Protease (40.6 mg active/g) | 0.8 | 0.6 | 0.7 | 0.9 | 0.7 |
| Mannanase: Mannaway ® | 0.07 | 0.05 | 0.045 | 0.06 | 0.04 |
| (25 mg active/g) | | | | | |
| Amylase: Stainzyme ® | 0.3 | 0 | 0.3 | 0.1 | 0 |
| (15 mg active/g) | | | | | |
| Amylase: Natalase ® | 0 | 0.2 | 0.1 | 0.15 | 0.07 |
| (29 mg active/g) | | | | | |
| Xyloglucanase | 0.2 | 0.1 | 0 | 0 | 0.05 |
| (Whitezyme ®, 20 mg | | | | | |
| active/g) | | | | | |
| Lipex ® (18 mg active/g) | 0.4 | 0.2 | 0.3 | 0.1 | 0.2 |
| Neat Perfume² | 0.5 | 0.5 | 0.5 | 0.5 | 0.5 |
| Ionic liquid System from IL | 3 | 2 | 1 | 0.5 | 1 |
| Examples 1 to 6 | | | | | |

| | |
|---|---|
| *Water, dyes & minors | Balance |

¹wt % relative to the total weight of the composition.
²Optional.

### Raw Materials and Notes for Detergent Composition Examples

1. “LAS” is linear alkylbenzenesulfonate having an average aliphatic carbon chain length C₉-C₁₅, supplier: Stepan, Northfield, Ill., USA or Huntsman Corp. (HLAS is acid form).
2. C₁₂₋₁₄ Dimethylhydroxyethyl ammonium chloride, supplier: Clariant GmbH, Germany.
3. AE3S is C₁₂₋₁₅ alkyl ethoxy (3) sulfate, supplier: Stepan, Northfield, Ill., USA.
4. AE7 is C₁₂₋₁₅ alcohol ethoxylate, with an average degree of ethoxylation of 7, supplier: Huntsman, Salt Lake City, Utah, USA
5. AES is C₁₀₋₁₈ alkyl ethoxy sulfate, supplier: Shell Chemicals.
6. AE9 is C₁₂₋₁₃ alcohol ethoxylate, with an average degree of ethoxylation of 9, supplier: Huntsman, Salt Lake City, Utah, USA
7. HSAS or HC1617HSAS is a mid-branched primary alkyl sulfate with average carbon chain length of about 16-17.
8. Sodium tripolyphosphate, supplier: Rhodia, Paris, France.
9. Zeolite A, supplier: Industrial Zeolite (U.K) Ltd, Grays, Essex, U.K.
10. 1.6R Silicate, supplier: Koma, Nestemica, Czech Republic
11. Sodium Carbonate, supplier: Solvay, Houston, Tex., USA.
12. Polyacrylate MW 4500, supplier: BASF, Ludwigshafen, Germany.
13. Carboxymethyl cellulose is Finnfix® V, supplier: CP Kelco, Arnhem, Netherlands.
14. Suitable chelants are, for example, diethylenetetraamine pentaacetic acid (DTPA), supplier: Dow Chemical, Midland, Mich., USA or Hydroxyethane di phosphonate (HEDP), supplier: Solutia, St Louis, Mo., USA Bagsvaerd, Denmark.
15. Savinase®, Natalase®, Stainzyme®, Lipex®, Celluclean™, Mannaway® and Whitezyme®, supplier: Novozymes, Bagsvaerd, Denmark.
16. Proteases, supplier: Genencor International, Palo Alto, Calif., USA (e.g. Purafect Prime®) or by Novozymes, Bagsvaerd, Denmark (e.g. Liquanase®, Coronase®).
17. Fluorescent Brightener 1 is Tinopal® AMS, Fluorescent Brightener 2 is Tinopal® CBS-X, Sulphonated zinc phthalocyanine and Direct Violet 9 is Pergasol® Violet BN-Z, supplier: Ciba Specialty Chemicals, Basel, Switzerland.
18. Sodium percarbonate, supplier: Solvay, Houston, Tex., USA.
19. Sodium perborate, supplier: Degussa, Hanau, Germany.
20. “NOBS” is sodium nonanoyloxybenzenesulfonate, supplier: Future Fuels, Batesville, USA.
21. “TAED” is tetraacetylethylenediamine (Peractive®), supplier: Clariant GmbH, Sulzbach, Germany.
22. “S-ACMC” is carboxymethylcellulose conjugated with C.I. Reactive Blue 19 (AZO-CM-CELLULOSE, product code S-ACMC), supplier: Megazyme, Wicklow, Ireland.
23. Soil release agent is Repel-o-Tex® PF, supplier: Rhodia, Paris, France.
24. Acrylic Acid/Maleic Acid Copolymer is molecular weight 70,000 and acrylate:maleate ratio 70:30, supplier: BASF, Ludwigshafen, Germany.
25. Na salt of Ethylenediamine-N,N′-disuccinic acid, (S,S) isomer (EDDS), supplier: Octel, Ellesmere Port, U.K.
26. Hydroxyethane di phosphonate (HEDP), supplier: Dow Chemical, Midland, Mich., USA.
27. Suds suppressor agglomerate, supplier: Dow Corning, Midland, Mich., USA.
28. HSAS is mid-branched alkyl sulfate as disclosed in U.S. Pat. Nos. 6,020,303 and 6,060,443.
29. C₁₂₋₁₄ dimethyl Amine Oxide, supplier: Procter & Gamble Chemicals, Cincinnati, USA.
30. Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40:60 and no more than 1 grafting point per 50 ethylene oxide units.
31. Ethoxylated polyethyleneimine is polyethyleneimine (MW=600) with 20 ethoxylate groups per —NH.
32. Cationic cellulose polymer is LK400, LR400 and/or JR30M, supplier: Amerchol Corporation, Edgewater N.J.
33. Note: all enzyme levels are expressed as % enzyme raw material.

### Example 4

### Hair Care Compositions

The following are non-limiting examples of hair care compositions containing ionic liquids of the present invention. They are prepared by admixture of the components described in Tables below, in the proportions indicated.

TABLE 7
Leave-on Conditioner Composition

| | Leave-on Conditioner |
|---|---|
| | Compositions (wt %) |
| Ingredients | Ex. 11 |
| Premix | — |
| Aminosilicone | — |
| PDMS | 1.0-1.5 |
| Gel matrix carrier | — |
| Behenyl trimethyl ammonium | — |
| chloride | |
| Stearamidopropyldimethylamine | 0.60-0.8 |
| (SAPDMA), C18 | |
| DTDMAC, C18 | 0.45-0.6 |
| (Quaternium-18) | |
| Citric Acid (anhydrous) | 0.10-0.25 |
| Cetyl alcohol | 0.80-1.0 |
| Stearyl alcohol | 0.54-1.0 |
| Deionized Water | Balance |
| Polymers | — |
| Hydroxyethylcellulose (HEC) | 0.15-0.50 |
| PEG-2M (Polyox WAR N-10) | 0.30-0.60 |
| Others | — |
| Ionic Liquid System from IL | 0.10-1.20 |
| Examples 1 to 6 | |
| Preservatives | 0.40-0.60 |

TABLE 8
Shampoo Compositions

| | Shampoo Compositions (wt %) | | |
|---|---|---|---|
| Ingredients | Ex. 12 | Ex. 13 | Ex. 14 |
| Water | q.s. | q.s. | q.s. |
| Polyquaternium 76¹ | 2.50 | — | — |
| Guar, Hydroxylpropyl Trimonium | — | 0.25 | — |
| Chloride² | | | |
| Polyquaterium 6³ | — | — | 0.79 |
| Sodium Laureth Sulfate (SLE3S)⁴ | 21.43 | 21.43 | 21.43 |
| Sodium Lauryl Sulfate (SLS)⁵ | 20.69 | 20.69 | 20.69 |
| Silicone⁶ | 0.75 | 1.00 | 0.5 |
| Cocoamidopropyl Betaine⁷ | 3.33 | 3.33 | 3.33 |
| Cocoamide MEA⁸ | 1.0 | 1.0 | 1.0 |
| Ethylene Glycol Distearate⁹ | 1.50 | 1.50 | 1.50 |
| Sodium Chloride¹⁰ | 0.25 | 0.25 | 0.25 |
| Free Perfume Oil | 0.70 | 0.70 | 0.70 |
| Ionic Liquid System from IL | 1.2 | 1.2 | 1.2 |
| Examples 1 to 6 | | | |
| Preservatives, pH adjusters | Up to 1% | Up to 1% | Up to 1% |

¹Mirapol AT-1, Copolymer of Acrylamide(AM) and TRIQUAT, MW = 1,000,000; CD = 1.6 meq./gram; 10% active, supplier: Rhodia.
²Jaguar C500, MW-500,000, CD = 0.7, supplier: Rhodia.
³Mirapol 100S, 31.5% active, supplier: Rhodia.
⁴Sodium Laureth Sulfate (SLS), 28% active, supplier: Procter & Gamble.
⁵Sodium Lauryl Sulfate, 29% active, supplier: Procter & Gamble.
⁶Glycidol Silicone VC2231-193C.
⁷Tegobetaine F-B, 30% active, supplier: Goldschmidt Chemicals.
⁸Monamid CMA, 85% active, supplier: Goldschmidt Chemicals.
⁹Ethylene Glycol Distearate, EGDS Pure, supplier: Goldschmidt Chemicals.
¹⁰Sodium Chloride USP (food grade), supplier: Morton. (Note that salt is an adjustable ingredient, higher or lower levels may be added to achieve target viscosity.)

TABLE 9
Shampoo Compositions (Cont.)

| | Shampoo | | |
|---|---|---|---|
| | Compositions (wt %) | | |
| Ingredients | Ex. 15 | Ex. 16 | Ex. 17 |
| Water | q.s. | q.s. | q.s. |
| Silicone A¹ | 1.0 | — | — |
| Silicone B² | — | 0.5 | — |
| Silicone C³ | — | — | 0.5 |
| Cyclopentasiloxane⁴ | — | 0.61 | 1.5 |
| Behenyl trimethyl ammonium | 2.25 | 2.25 | 2.25 |
| chloride⁵ | | | |
| Isopropyl alcohol | 0.60 | 0.60 | 0.60 |
| Cetyl alcohol⁶ | 1.86 | 1.86 | 1.86 |
| Stearyl alcohol⁷ | 4.64 | 4.64 | 4.64 |
| Disodium EDTA | 0.13 | 0.13 | 0.13 |
| NaOH | 0.01 | 0.01 | 0.01 |
| Benzyl alcohol | 0.40 | 0.40 | 0.40 |
| Methylchloroisothiazolinone/ | 0.0005 | 0.0005 | 0.0005 |
| Methylisothiazolinone⁸ | | | |
| Panthenol⁹ | 0.10 | 0.10 | 0.10 |
| Panthenyl ethyl ether¹⁰ | 0.05 | 0.05 | 0.05 |
| Free Perfume Oil | 0.35 | 0.35 | 0.35 |
| Ionic Liquid System from IL | 1.2 | 1.2 | 1.2 |
| Examples 1 to 6 | | | |

¹Glycidol Silicone VC2231-193.
²Glycidol Silicone VC2231-193F.
³Glycidol Silicone VC2231-193A.
⁴Cyclopentasiloxane: SF1202, supplier: Momentive Performance Chemicals.
⁵Behenyl trimethyl ammonium chloride/Isopropyl alcohol: Genamin ™ KMP, supplier: Clariant.
⁶Cetyl alcohol: Konol ™ series, supplier: Shin Nihon Rika.
⁷Stearyl alcohol: Konol ™ series, supplier: Shin Nihon Rika.
⁸Methylchloroisothiazolinone/Methylisothiazolinone: Kathon ™ CG, supplier: Rohm & Haas.
⁹Panthenol, supplier: Roche.
¹⁰Panthenyl ethyl ether, supplier: Roche.

TABLE 10
Shampoo Compositions (Cont.)

Shampoo
Compositions (wt %)

| Ingredients | Ex. 18 | Ex. 19 |
|---|---|---|
| Sodium Laureth Sulfate | 10.00 | 10.00 |
| Sodium Lauryl Sulfate | 1.50 | 1.50 |
| Cocamidopropyl betaine | 2.00 | 2.00 |
| Guar Hydroxypropyl trimonium | 0.40 | — |
| chloride¹ | | |
| Guar Hydroxypropyl trimonium | — | 0.40 |
| chloride² | | |
| Dimethicone³ | 2.00 | 2.00 |
| Gel Network⁴ | — | 27.27 |
| Ethylene Glycol Distearate | 1.50 | 1.50 |
| 5-Chloro-2-methy1-4-isothiazolin- | 0.0005 | 0.0005 |
| 3-one, Kathon CG | | |
| Sodium Benzoate | 0.25 | 0.25 |
| Disodium EDTA | 0.13 | 0.13 |
| Free Perfume Oil | 0.70 | 0.70 |
| Ionic Liquid System from | 1.0 | 1.0 |
| Examples 1 to 6 | | |
| Citric Acid/Sodium Citrate | pH q.s. | pH q.s. |
| Dihydrate | | |
| Sodium Chloride/Ammonium | visc. q.s. | visc. q.s. |
| Xylene Sulfonate | | |
| Water | q.s. | q.s. |

¹Jaguar C17, supplier: Rhodia.
²N-Hance 3269 (with Mol. W. of ~500,000 and 0.8 meq/g), supplier: Aqulaon/Hercules.
³Viscasil 330M, supplier: General Electric Silicones.
⁴Gel Networks; See Composition below. The water is heated to about 74° C. and the Cetyl Alcohol, Stearyl Alcohol, and the SLES Surfactant are added to it. After incorporation, this mixture is passed through a heat exchanger where it is cooled to about 35° C. As a result of this cooling step, the Fatty Alcohols and surfactant crystallized to form a crystalline gel network.

| Ingredients | Wt. % |
|---|---|
| Water | 86.14% |
| Cetyl Alcohol | 3.46% |
| Steary Alcohol | 6.44% |
| Sodium laureth-3 sulfate (28% Active) | 3.93% |
| 5-Chloro-2-methyl-4-isothiazolin-3-one, Kathon CG | 0.03% |

### Example 5

### Lotion Compositions

The following are non-limiting examples of ionic liquids in lotion compositions containing ionic liquids of the present invention. For the examples described in Table 11, in a suitable container, combine the ingredients of Phase A. In a separate suitable container, combine the ingredients of Phase B. Heat each phase to 73-78° C. while mixing each phase using a suitable mixer (e.g., Anchor blade, propeller blade, or IKA T25) until each reaches a substantially constant desired temperature and is homogenous. Slowly add Phase B to Phase A while continuing to mix Phase A. Continue mixing until batch is uniform. Pour product into suitable containers at 73-78° C. and store at room temperature. Alternatively, continuing to stir the mixture as temperature decreases results in lower observed hardness values at 21 OC and 33° C.

TABLE 11
Lotion Compositions

| | Lotion Compositions | | |
|---|---|---|---|
| | (wt %) | | |
| Ingredients | Ex. 20 | Ex. 21 | Ex. 22 |

PHASE A

| | | | |
|---|---|---|---|
| DC-9040¹ | 8.60 | 3.00 | 5.00 |
| Dimethicone | 4.09 | 4.00 | 4.00 |
| Polymethylsilsesquioxane² | 4.09 | 4.00 | 4.00 |
| Cyclomethicone | 11.43 | 0.50 | 11.33 |
| KSG-210³ | 5.37 | 5.25 | 5.40 |
| Polyethylene wax⁴ | 3.54 | — | 2.05 |
| DC-2503 Cosmetic Wax⁵ | 7.08 | 10.00 | 3.77 |
| Hydrophobic TiO₂ | — | — | 0.50 |
| Iron oxide coated Mica | — | — | 0.65 |
| TiO₂ Coated Mica | 1.00 | 1.00 | — |
| Ionic Liquid System from IL | 1.00 | 1.00 | 1.00 |
| Examples 1 to 6 | | | |

PHASE B

| | | | |
|---|---|---|---|
| Glycerin | 10.00 | 10.00 | 10.00 |
| Dexpanthenol | 0.50 | 0.50 | 0.50 |
| Pentylene Glycol | 3.00 | 3.00 | 3.00 |
| Hexamidine Diisethionate⁶ | 0.10 | 0.10 | 0.10 |
| Niacinamide⁷ | 5.00 | 5.00 | 5.00 |
| Methylparaben | 0.20 | 0.20 | 0.20 |
| Ethylparaben | 0.05 | 0.05 | 0.05 |
| Sodium Citrate | 0.20 | 0.20 | 0.20 |
| Citric Acid | 0.03 | 0.03 | 0.03 |
| Sodium Benzoate | 0.05 | 0.05 | 0.05 |
| Sodium Chloride | 0.50 | 0.50 | 0.50 |
| FD&C Red #40 (1%) | 0.05 | 0.05 | 0.05 |
| Water | q.s. | q.s. | q.s. |
| Hardness at 21° C. (g) | 33.3 | 15.4 | 14.2 |
| Hardness at 33° C. (g) | 6.4 | 0.7 | 4.0 |

¹12.5% Dimethicone Crosspolymer in Cyclopentasiloxane, supplier: Dow Corning.
²Tospearl ™ 145A or Tospearl 2000, supplier: GE Toshiba Silicone.
³25% Dimethicone PEG-10/15 Crosspolymer in Dimethicone, supplier: Shin-Etsu.
⁴Jeenate ™ 3H polyethylene wax, supplier: Jeen.
⁵Stearyl Dimethicone, supplier: Dow Corning.
⁶Hexamidine diisethionate, available from Laboratoires Serobiologiques.
⁷Additionally or alternatively, the composition may comprise one or more other skin care actives, their salts and derivatives, as disclosed herein, in amounts also disclosed herein as would be deemed suitable by one of skill in the art.

### Example 6

### Antiperspirant/Deodorant Compositions

The following are non-limiting examples of antiperspirant/deodorant compositions containing ionic liquids of the present invention. The below examples in Table 12 can be made via the following general process, which one skilled in the art will be able to alter to incorporate available equipment. The ingredients of Part I and Part II are mixed in separate suitable containers. Part II is then added slowly to Part I under agitation to assure the making of a water-in-silicone emulsion. The emulsion is then milled with suitable mill, for example a Greeco 1L03 from Greeco Corp, to create a homogenous emulsion. Part III is mixed and heated to 88° C. until the all solids are completely melted. The emulsion is then also heated to 88° C. and then added to the Part III ingredients. The final mixture is then poured into an appropriate container, and allowed to solidify and cool to ambient temperature.

TABLE 12
Antiperspirant/Deodorant Compositions

| | Antiperspirant/Deodorant | | | | |
|---|---|---|---|---|---|
| | Compositions (wt %¹) | | | | |
| Ingredients | Ex. 23 | Ex. 24 | Ex. 25 | Ex. 26 | Ex. 27 |

Part I: Partial Continuous Phase

| | | | | | |
|---|---|---|---|---|---|
| Hexamethyldisiloxane¹ | 22.65 | 21.25 | 21.25 | 21.25 | 21.25 |
| DC5200² | 1.20 | 1.20 | 1.20 | 1.20 | |
| Fragrance | 0.35 | 1.25 | 1.25 | 1.25 | 1.25 |
| Ionic Liquid System | 1.00 | 1.00 | 1.00 | 1.00 | 1.00 |
| from IL Examples 1 to 6 | | | | | |
| Shin Etsu KF 6038³ | — | — | — | — | 1.20 |

Part II: Disperse Phase

| | | | | | |
|---|---|---|---|---|---|
| ACH (40% solution)⁴ | 40.00 | 55.0 | — | — | — |
| IACH (34% solution)⁵ | — | 2.30 | 49.00 | — | — |
| ZAG (30% solution)⁶ | — | — | — | 52.30 | 52.30 |
| propylene glycol | 5.00 | — | 5.00 | 5.00 | 5.00 |
| Water | 12.30 | — | 3.30 | — | — |

Part III: Structurant Plus Remainder of Continuous Phase

| | | | | | |
|---|---|---|---|---|---|
| FinSolve TN | 6.50 | 6.00 | 6.50 | 6.00 | 6.50 |
| Ozocrite Wax | — | — | — | | 12.00 |
| Performalene PL⁷ | 11.00 | 11.00 | 12.00 | 12.00 | |
| Aqueous Phase | 37.7 | 79.5 | 40.5 | 60.3 | 60.3 |
| Conductivity (mS/cm) | | | | | |

¹DC 246 fluid, supplier: Dow Corning.
²Supplier: Dow Corning.
³Supplier: Shinetsu.
⁴Standard aluminum chlorohydrate solution.
⁵IACH solution stabilized with calcium.
⁶IZAG solution stabilized with calcium.
⁷Supplier: New Phase Technologies.

Examples 28-31 in Table 13 can be made as follows whereby all ingredients except the fragrance, linalool, and dihydromyrcenol are combined in a suitable container and heated to about 85° C. to form a homogenous liquid. The solution is then cooled to about 62° C. and then the fragrance, linalool, and dihydromyrcenol are added. The mixture is then poured into an appropriate container and allowed to solidify up cooling to ambient temperature.

Example 32 in Table 13 can be made as follows whereby all the ingredients except the propellant are combined in an appropriate aerosol container. The container is then sealed with an appropriate aerosol delivery valve. Next air in the container is removed by applying a vacuum to the valve and then propellant is added to container through the valve. Finally an appropriate actuator is connected to the valve to allow dispensing of the product.

TABLE 13
Antiperspirant/Deodorant Compositions (Cont.)

| | Antiperspirant/Deodorant | | | | |
|---|---|---|---|---|---|
| | Compositions (wt %¹) | | | | |
| Ingredients | Ex. 28 | Ex. 29 | Ex. 30 | Ex. 31 | Ex. 32 |
| Product Form | Solid | Solid | Solid | Solid | Solid or |
| | | | | | Spray |
| Dipropylene glycol | 45 | 22 | 20 | 30 | 20 |
| Propylene glycol | 22 | 45 | 22 | — | — |
| Tripopylene glycol | — | — | 25 | — | — |
| Glycerine | — | — | — | 10 | — |
| PEG-8 | — | — | — | 20 | — |
| Ethanol | — | — | — | — | q.s. |
| Water | q.s. | q.s. | q.s. | q.s. | — |
| Sodium stearate | 5.5 | 5.5 | 5.5 | 5.5 | — |
| Tetra sodium EDTA | 0.05 | 0.05 | 0.05 | 0.05 | — |
| Sodium hydroxide | 0.04 | 0.04 | 0.04 | 0.04 | — |
| Triclosan | 0.3 | 0.3 | 0.3 | 0.3 | — |
| Fragramce | 0.5 | 0.5 | 0.5 | 0.5 | 0.5 |
| Ionic Liquid System | 1.0 | 1.0 | 1.0 | 1.0 | 0.5 |
| from IL Examples 1 to 6 | | | | | |
| Dihydromyrcenol | 0.3 | 0.1 | 0.3 | 0.5 | 0.1 |
| Linalool | 0.2 | 0.15 | 0.2 | 0.25 | 0.15 |
| Propellant (1,1 | — | — | — | — | 40 |
| difluoroethane) | | | | | |

### Example 7

### Rinse-Off Conditioner Compositions

The following are non-limiting examples of rinse-off conditioner compositions containing ionic liquids of the present invention. Examples 33 and 35-38 in Table 14 are prepared as follows: cationic surfactants, high melting point fatty compounds are added to water with agitation, and heated to about 80° C. The mixture is cooled down to about 50° C. to form a gel matrix carrier. Separately, slurries of ionic liquids and silicones are mixed with agitation at room temperature to form a premix. The premix is added to the gel matrix carrier with agitation. If included, other ingredients such as preservatives are added with agitation. Then the compositions are cooled down to room temperature.

Example 34 in Table 14 is prepared as follows: cationic surfactants, high melting point fatty compounds are added to water with agitation, and heated to about 80° C. The mixture is cooled down to about 50° C. to form a gel matrix carrier. Then, silicones are added with agitation. Separately, ionic liquids, and if included, other ingredients such as preservatives are added with agitation. Then the compositions are cooled down to room temperature.

TABLE 14
Rinse-Off Conditioner Compositions

| | Rinse-Off Conditioner Compositions (wt % ¹) | | | | | |
|---|---|---|---|---|---|---|
| | Ex. | Ex. | Ex. | Ex. | Ex. | Ex. |
| Ingredients | 33 | 34 | 35 | 36 | 37 | 38 |

Pre-Mix

| | | | | | | |
|---|---|---|---|---|---|---|
| Aminosilicone-1 ¹ | 0.50 | 0.50 | — | — | — | — |
| Aminosilicone-2 ² | — | — | 0.50 | 0.50 | 0.50 | — |
| PDMS | — | — | — | — | — | 0.50 |
| Ionic Liquid | — | 1.0 | 1.0 | 1.0 | 1.0 | 1.0 |
| System from IL | | | | | | |
| Examples 1 to 6 | | | | | | |

Gel Matrix Carrier

| | | | | | | |
|---|---|---|---|---|---|---|
| Behenyl trimethyl | 2.30 | 2.30 | 2.30 | 2.30 | 2.30 | 2.30 |
| ammonium chloride | | | | | | |
| Cetyl alcohol | 1.5 | 1.5 | 1.5 | 1.5 | 1.5 | 1.5 |
| Stearyl alcohol | 3.8 | 3.8 | 3.8 | 3.8 | 3.8 | 3.8 |
| Deionized Water | q.s. | q.s. | q.s. | q.s. | q.s. | q.s. |
| Preservatives | 0.4 | 0.4 | 0.4 | 0.4 | 0.4 | 0.4 |
| Panthenol | — | — | 0.03 | — | — | — |
| Panthenyl | — | — | 0.03 | — | — | — |
| ethyl ether | | | | | | |

¹ Aminosilicone-1 (AMD): having an amine content of 0.12-0.15 m mol/g and a viscosity of 3,000-8,000 mPa · s, which is water insoluble.
² Aminosilicone-2 (TAS): having an amine content of 0.04-0.06 m mol/g and a viscosity of 10,000-16,000 mPa · s, which is water insoluble.

### Example 8

### Body Cleansing Compositions

The following are non-limiting examples of body cleansing compositions containing ionic liquids of the present invention. They are prepared by admixture of the components described in Table 15, in the proportions indicated.

TABLE 15
Lotion Compositions

| | Lotion Compositions (wt %) | | |
|---|---|---|---|
| Ingredients | Ex. 39 | Ex. 40 | Ex. 41 |

Part I: Cleansing Phase Composition

| | | | |
|---|---|---|---|
| Sodium Trideceth Sulfate | 5.9 | 5.9 | 5.9 |
| (sulfated from Iconol TDA-3 | | | |
| to >95% sulfate)¹ | | | |
| Sodium Lauryl Sulfate² | 5.9 | 5.9 | 5.9 |
| Sodium Lauroamphoacetate³ | 3.6 | 3.6 | 3.6 |
| Guar Hydroxypropyltrimonium | — | 0.3 | 0.7 |
| Chloride⁴ | | | |
| Guar Hydroxypropyltrimonium | 0.6 | — | — |
| Chloride⁵ | | | |
| Stabylen 30⁶ | 0.33 | 0.33 | 0.33 |
| Sodium Chloride | 3.75 | 3.75 | 3.75 |
| Trideceth-3⁷ | 1.75 | 1.75 | 1.75 |
| Methyl chloro isothiazolinone | 0.033 | 0.033 | 0.033 |
| and methyl isothiazolinone⁸ | | | |
| EDTA⁹ | 0.15 | 0.15 | 0.15 |
| Sodium Benzoate | 0.20 | 0.20 | 0.20 |
| Citric Acid, titrate | pH 5.7 ± | pH 5.7 ± 0.2 | pH 5.7 ± 0.2 |
| | 0.2 | | |
| Perfume | 1.11% | 1.11% | 1.11% |
| Water and Minors (NaOH) | q.s. | q.s. | q.s. |

Part II: Benefit Phase Composition

| | | | |
|---|---|---|---|
| Petrolatum¹⁰ | 60 | 60 | 60 |
| Mineral oil¹¹ | 20 | 20 | 20 |
| Ionic Liquid System from | 10 | 10 | 10 |
| Examples 1 to 6 | | | |

¹Supplier: BASF.
²Supplier: Procter & Gamble.
³Supplier: Cognis Chemical Corp.
⁴N-Hance 3196, supplier: Aqualon.
⁵Jaguar C-17, supplier: Rhodia.
⁶Acrylates/Vinyl Isodecanoate, 3V.
⁷Iconal TDA-3, supplier: BASF.
⁸Kathon CG, supplier: Rohm & Haas.
⁹Dissolvine NA 2x.
¹⁰G2218, supplier: Sonnerbonn.
¹¹Hydrobrite 1000, supplier: Sonnerbonn.

### Example 9

### Fragrance Compositions

The following are non-limiting examples of fragrance compositions containing ionic liquids of the present invention. They are prepared by admixture of the components described in Table 16, in the proportions indicated.

TABLE 16
Fragrance Compositions

| | Fragrance Compositions (wt %) | | | | |
|---|---|---|---|---|---|
| Ingredients | Ex. 42 | Ex. 43 | Ex. 44 | Ex. 45 | Ex. 46 |
| Fragrance material | 5.0 | 5.0 | 5.0 | 1.0 | 1.0 |
| Dipropylene glycol¹ | 20.0 | 33.0 | 40.0 | — | 33.0 |
| Ionic Liquid System | 75.0 | 45.0 | 44.0 | 99.0 | 49.0 |
| from IL Examples 1 to 6 | | | | | |
| Ethanol | — | 17 | — | — | 17.0 |
| Demineralized water | — | — | 11.- | — | — |

¹Supplier: Sigma Aldrich.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An ionic liquid system comprising one or more ionic liquids, each comprising of a cation and an anion, wherein the conjugate acid of at least one of the anions is a perfume raw material with a pKₐ from about 0 to about 14.

2. The ionic liquid system according to claim 1, wherein each ionic liquid comprises a cation independently selected from the group consisting of: 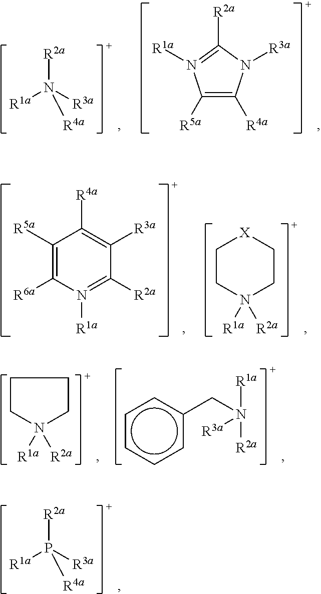
and combinations thereof;
X is CH₂ or O;
each R^{1a}, R^{3a}, and R^{4a} are independently selected from hydrogen, C₁-C₂₀ alkyl, C₁-C₂₀ alkenyl, C₁-C₂₀ alkynyl, C₁-C₂₀ alkoxy, C₁-C₂₀ alkoxyC₁-C₂₀alkyl, C₃-C₇ cycloalkyl, C₃-C₇ cycloalkylC₁-C₄alkyl, C₂-C₂₀ heterocyclyl, C₆-C₁₀ aryl, C₆-C₁₀ arylC₁-C₁₀alkyl, C₁-C₁₀ heteroaryl, halo, haloC₁-C₂₀alkyl, hydroxyl, hydroxyC₁-C₂₀alkyl, or —N(R^{6a})₂;
each R^{2a} is independently selected from hydrogen, C₁-C₂₀ alkyl, C₁-C₂₀ alkenyl, or C₁-C₂₀ alkynyl;
each R^{5a} is independently selected from hydrogen, C₁-C₂₀ alkyl, C₁-C₂₀ alkenyl, C₁-C₂₀ alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, alkylene chain, alkenylene chain, or alkynylene chain; and
each R^{8a} is independently selected from a hydrogen, alkylene chain, alkenylene chain, or alkynylene chain.

3. The ionic liquid system according to claim 2, wherein each of the cations is independently selected from the group consisting of: 1-butyl-3-methylimidazolium; (N-ethyl-2-(2-methoxyethoxy)-N,N-dimethylethanaminium); 2-(2-ethoxyethoxy)-N-ethyl-N,N-dimethylethanaminium; N-benzyl-N,N-dimethyloctan-1-aminium; N-benzyl-N,N-dimethylnonan-1-aminium; and combinations thereof.

4. The ionic liquid system according to claim 1, wherein the conjugate acid of at least one of the anions is a perfume raw material having a c Log P value between about 0 to about 7.

5. The ionic liquid system system according to claim 4, wherein the conjugate acid of at least one of the anions is a perfume raw material selected from the group consisting of: benzoic acid; benzeneacetic acid; 4-methoxybenzoic acid; 2-propenoic acid, 3-phenyl-(2E)-2-methyl-2-pentenoic acid; benzenepropanoic acid; decanoic acid; octanoic acid; dodecanoic acid; 5-decenoic acid; 3-ethoxy-4-hydroxybenzaldehyde; 4-hydroxy-3-methoxybenzaldehyde; 7-hydroxy-2H-1-benzopyran-2-one; 1-(4-hydroxyphenyl) butan-1-one; 4-hydroxy-3-methoxybenzoic acid; 4H-pyran-4-one, 2-ethyl-3-hydroxy-; 3-hydroxy-2-methyl-4-pyrone; 3-(4-hydroxy-3-methoxyphenyl)prop-2-enal; 3-methoxy-5-methylphenol; 4-chloro-3,5-dimethylphenol; 5-methyl-2-(2-sulfanylpropan-2-yl)cyclohexan-1-one; 4-(4-hydroxyphenyl)butan-2-one; 4-(4-hydroxy-3-methoxyphenyl)butan-2-one; (2-Hydroxy-4-methoxyphenyl)-phenylmethanone; pentyl 2-hydroxybenzoate; butyl 2-hydroxybenzoate; [(Z)-hex-3-enyl] 2-hydroxybenzoate; cyclohexyl 2-hydroxybenzoate; ethyl 2-hydroxybenzoate; 3-methylbutyl 2-hydroxybenzoate; 2-methylpropyl 2-hydroxybenzoate; methyl 2-hydroxybenzoate; hexyl 2-hydroxybenzoate; 3-methylbut-2-enyl 2-hydroxybenzoate; benzyl 2-hydroxybenzoate; triethyl 2-hydroxypropane-1,2,3-tricarboxylate; 2,4-dimethylphenol; 4-ethyl-2-methoxyphenol; 4-ethenylphenol; 2-methyl-5-propan-2-ylphenol; 2-methoxy-4-[(Z)-prop-1-enyl]phenol; 2-methoxy-4-methylphenol; 2-methoxy-4-propylphenol; 2-methoxy-4-prop-2-enylphenol; 2-methoxyphenol; phenol; 2-methoxy-4-prop-1-en-2-ylphenol; 3-ethylphenol; 2-methylphenol; 4-methylphenol; 4-ethylphenol; 4-tert-butylphenol; 2-methoxy-4-[(E)-prop-1-enyl]phenol; 2-ethoxy-4-methylphenol; 2-ethoxy-5-prop-1-enylphenol; phenol, 4-(3,6-dihydro-4-methyl-2H-pyran-2-yl)-2-methoxy-; phenol, 2-methoxy-4-(tetrahydro-4-methylene-2H-pyran-2-yl)-; 2-ethoxy-4-(methoxymethyl)phenol; 2-methoxy-4-(methoxymethyl)phenol; methyl 2,4-dihydroxy-3,6-dimethylbenzoate; ethyl 3-oxobutanoate; 3-methylbutyl 3-oxobutanoate; 5-methyl-2-propan-2-ylphenol; 2,6-ditert-butyl-4-methylphenol; 2-hydroxy-1,2-diphenylethanone; diethyl propanedioate; ethyl 2-acetyloctanoate; (1-acetyloxy-3-hydroxypropan-2-yl) acetate; 3-hydroxybutan-2-one; and combinations thereof.

6. The ionic liquid system according to claim 5, wherein the conjugate acid of at least one of the anions is independently a perfume raw material selected from the group consisting of: benzoic acid; benzeneacetic acid; 4-methoxybenzoic acid; 2-propenoic acid, 3-phenyl-(2E)-2-methyl-2-pentenoic acid; benzenepropanoic acid; decanoic acid; octanoic acid; dodecanoic acid; 5-decenoic acid; 3-ethoxy-4-hydroxybenzaldehyde; 4-hydroxy-3-methoxybenzaldehyde; 7-hydroxy-2H-1-benzopyran-2-one; and combinations thereof, preferably 3-ethoxy-4-hydroxybenzaldehyde; 4-hydroxy-3-methoxybenzaldehyde; and combinations thereof.

7. The ionic liquid system according to claim 1, wherein the system comprises two, three, four, five or more ionic liquids.

8. The ionic liquid system according to claim 7, wherein at least one of the ionic liquids comprises anions whose conjugate acids are not perfume raw materials, and are independently selected from the group consisting of:
[R¹—O—C(O).CH(SO₃)R³—C(O).O—R²]⁻ (I)
(a)
wherein:
R¹ and R² are independently selected from the group consisting of alkyl or alkenyl, provided that the alkyl is not substituted with nitro, azido or halide; and
R³ is alkylene, heteroarylene, arylene, or cycloalkylene;
(b) 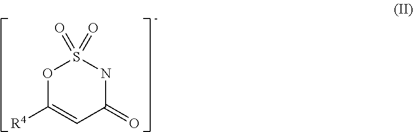
wherein:
R⁴ is selected from the group consisting of hydrogen, cyano, alkyl, alkoxy and alkoxyalkyl;
(c) 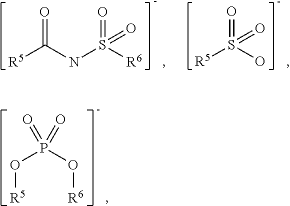
bistriflamide and combinations thereof;
wherein:
each R⁵ and R⁶ are independently selected from the group consisting of hydrogen, alkyl, alkenyl, alkynyl, alkoxyalkyl, cycloalkyl, cycloalkylalkyl, aryl, aralkyl, heterocyclyl, heterocyclyalkyl, heteroaryl and heteroarylalkyl; and
(d) combinations thereof.

9. The ionic liquid system according to claim 8, wherein the anions whose conjugate acids are not perfume raw materials are independently selected from the group consisting of: 1,4-bis(2-ethylhexoxy)-1,4-dioxobutane-2-sulfonate; 6-methyl-3,4-dihydro-1,2,3-oxathiazin-4-one 2,2-dioxide; and combinations thereof.

10. The ionic liquid system according to claim 1, wherein the ionic liquid system further comprises a perfume microcapsule comprising from about 1% w/w to about 90% w/w, based on the total perfume microcapsule weight, of one or more perfume raw materials.

11. The ionic liquid system according to claim 10, wherein the perfume raw materials comprise materials selected from the group consisting of:
a) a perfume composition having a C log P of less than 4.5;
b) a perfume composition comprising, based on total perfume composition weight, 60% perfume materials having a C log P of less than 4.0;
c) a perfume composition comprising, based on total perfume composition weight, 35% perfume materials having a C log P of less than 3.5;
d) a perfume composition comprising, based on total perfume composition weight, 40% perfume materials having a C log P of less than 4.0 and at least 1% perfume materials having a C log P of less than 2.0;
e) a perfume composition comprising, based on total perfume composition weight, 40% perfume materials having a C log P of less than 4.0 and at least 15% perfume materials having a C log P of less than 3.0;
f) a perfume composition comprising, based on total perfume composition weight, at least 1% butanoate esters and at least 1% of pentanoate esters;
g) a perfume composition comprising, based on total perfume composition weight, at least 2% of an ester comprising an allyl moiety and at least 10% of another perfume comprising an ester moiety;
h) a perfume composition comprising, based on total perfume composition weight, at least 1% of an aldehyde comprising an alkyl chain moiety;
i) a perfume composition comprising, based on total perfume composition weight, at least 2% of a butanoate ester;
j) a perfume composition comprising, based on total perfume composition weight, at least 1% of a pentanoate ester;
k) a perfume composition comprising, based on total perfume composition weight, at least 3% of an ester comprising an allyl moiety and 1% of an aldehyde comprising gan alkyl chain moiety;
l) a perfume composition comprising, based on total perfume composition weight, at least 25% of a perfume comprising an ester moiety and 1% of an aldehyde comprising an alkyl chain moiety;
m) a perfume composition comprising, based on total perfume composition weight, at least 2% of a material selected from the group consisting of 4-(2,6,6-trimethyl-1-cyclohexenyl)-3-buten-2-one; 4-(2,6,6-trimethyl-2-cyclohexenyl)-3-buten-2-one; 3-buten-2-one,3-methyl-4-(2,6,6-trimethyl-1-cyclohexen-2-yl)-; and mixtures thereof;
n) a perfume composition comprising, based on total perfume composition weight, at least 0.1% of tridec-2-enonitrile; mandaril; or mixtures thereof;
o) a perfume composition comprising, based on total perfume composition weight, at least 2% of a material selected from 3,7-dimethyl-6-octenenitrile; 2-cyclohexylidene-2-phenylacetonitrile; or mixtures thereof;
p) a perfume composition comprising, based on total perfume composition weight, at least 80% of one or more perfumes comprising a moiety selected from the group consisting of esters, aldehydes, ionones, nitriles, ketones and combinations thereof;
q) a perfume composition comprising, based on total perfume composition weight, at least 3% of an ester comprising an allyl moiety; a perfume composition comprising, based on total perfume composition weight, at least 20% of a material selected from the group consisting of: 1-methylethyl-2-methylbutanoate; ethyl-2-methyl pentanoate; 1,5-dimethyl-1-ethenylhexyl-4-enyl acetate; p-meth-1-en-8-yl acetate; 4-(2,6,6-trimethyl-2-cyclohexenyl)-3-buten-2-one; 4-acetoxy-3-methoxy-1-propenylbenzene; 2-propenyl cyclohexanepropionate; bicyclo[2.2.1]hept-5-ene-2-carboxylic acid, 3-(1-methylethyl)-ethyl ester; bycyclo[2.2.1]heptan-2-ol, 1,7,7-trimethyl-, acetate; 1,5-dimethyl-1-ethenylhex-4-enylacetate; hexyl 2-methyl propanoate; ethyl-2-methylbutanoate; 4-undecanone; 5-heptyldihydro-2(3h)-furanone; 1,6-nonadien-3-ol,3,7dimethyl-; 3,7-dimethylocta-1,6-dien-3-o; 3-cyclohexene-1-carboxaldehyde,dimethyl-; 3,7-dimethyl-6-octene nitrile; 4-(2,6,6-trimethyl-1-cyclohexenyl)-3-buten-2-one; tridec-2-enonitrile; patchouli oil; ethyl tricycle [5.2.1.0]decan-2-carboxylate; 2,2-dimethyl-cyclohexanepropanol; hexyl ethanoate, 7-acetyl,1,2,3,4,5,6,7,8-octahydro-1,1,6,7-tetramethyl naphtalene; allyl-cyclohexyloxy acetate; methyl nonyl acetic aldehyde; 1-spiro[4,5]dec-7-en-7-yl-4-pentenen-1-one; 7-octen-2-ol,2-methyl-6-methylene-,dihydro; cyclohexanol,2-(1,1-dimethylethyl)-, acetate; hexahydro-4,7-methanoinden-5(6)-yl propionatehexahydro-4,7-methanoinden-5(6)-yl propionate; 2-methoxynaphtalene; 1-(2,6,6-trimethyl-3-cyclohexenyl)-2-buten-1-one; 1-(2,6,6-trimethyl-2-cyclohexenyl)-2-buten-1-one; 3,7-dimethyloctan-3-ol; 3-buten-2-one,3-methyl-4-(2,6,6-trimehtyl-1-cyclohexen-2-yl)-; hexanoic acid, 2-propenyl ester; (Z)-non-6-en-1-al; 1-decyl aldehyde; 1-octanal; 4-t-butyl-α-methylhydrocinnamaldehyde; α-hexylcinnamaldehyde; ethyl-2,4-hexadienoate; 2-propenyl 3-cyclohexanepropanoate; and mixtures thereof;
r) a perfume composition comprising, based on total perfume composition weight, at least 20% of a material selected from the group consisting of: 1-methylethyl-2-methylbutanoate; ethyl-2-methyl pentanoate; 1,5-dimethyl-1-ethenylhex-4-enyl acetate; p-menth-1-en-8-yl acetate; 4-(2,6,6-trimethyl-2-cyclohexenyl)-3-buten-2-one; 4-acetoxy-3-methoxy-1-propenylbenzene; 2-propenyl cyclohexanepropionate; bicyclo[2.2.1]hept-5-ene-2-carboxylic acid,3-(1-methylethyl)-ethyl ester; bycyclo [2.2.1]heptan-2-ol, 1,7,7-trimethyl-, acetate; 1,5-dimethyl-1-ethenylhex-4-enyl acetate; hexyl 2-methyl propanoate; ethyl-2-methylbutanoate,4-undecanolide; 5-heptyldihydro-2(3h)-furanone; 5-hydroxydodecanoic acid; decalactones; undecalactones, 1,6-nonadien-3-ol,3,7dimethyl-; 3,7-dimethylocta-1,6-dien-3-ol; 3-cyclohexene-1-carboxaldehyde,dimethyl-; 3,7-dimethyl-6-octene nitrile; 4-(2,6,6-trimethyl-1-cyclohexenyl)-3-buten-2-one; tridec-2-enonitrile; patchouli oil; ethyl tricycle [5.2.1.0]decan-2-carboxylate; 2,2-dimethyl-cyclohexanepropanol; allyl-cyclohexyloxy acetate; methyl nonyl acetic aldehyde; 1-spiro[4,5]dec-7-en-7-yl-4-pentenen-1-one; 7-octen-2-ol,2-methyl-6-methylene-,dihydro, cyclohexanol,2-(1,1-dimethylethyl)-, acetate; hexahydro-4,7-methanoinden-5(6)-yl propionatehexahydro-4,7-methanoinden-5(6)-yl propionate; 2-methoxynaphtalene; 1-(2,6,6-trimethyl-3-cyclohexenyl)-2-buten-1-one; 1-(2,6,6-trimethyl-2-cyclohexenyl)-2-buten-1-one; 3,7-dimethyloctan-3-ol; 3-buten-2-one,3-methyl-4-(2,6,6-trimehtyl-1-cyclohexen-2-yl)-; hexanoic acid, 2-propenyl ester; (Z)-non-6-en-1-al; 1-decyl aldehyde; 1-octanal; 4-t-butyl-α-methylhydrocinnamaldehyde; ethyl-2,4-hexadienoate; 2-propenyl 3-cyclohexanepropanoate; and mixtures thereof;
s) a perfume composition comprising, based on total perfume composition weight, at least 5% of a material selected from the group consisting of 3-cyclohexene-1-carboxaldehyde,dimethyl-; 3-buten-2-one,3-methyl-4-(2,6,6-trimethyl-1-cyclohexen-2-yl)-; patchouli oil; Hexanoic acid, 2-propenyl ester; 1-Octanal; 1-decyl aldehyde; (Z)-non-6-en-1-al; methyl nonyl acetic aldehyde; ethyl-2-methylbutanoate; 1-methylethyl-2-methylbutanoate; ethyl-2-methyl pentanoate; 4-hydroxy-3-ethoxybenzaldehyde; 4-hydroxy-3-methoxybenzaldehyde; 3-hydroxy-2-methyl-4-pyrone; 3-hydroxy-2-ethyl-4-pyrone; and mixtures thereof;
t) a perfume composition comprising, based on total perfume composition weight, less than 10% perfume materials having a C log P greater than 5.0;
u) a perfume composition comprising geranyl palmitate;
v) a perfume composition comprising a first and an optional second material, the first material having:
(i) a C log P of at least 2; and
(ii) a boiling point of less than about 280° C.;
wherein, the optional second material, if present, having a C log P of less than 2.5; and
w) combinations thereof.

12. An ionic liquid system comprising Σ(I_{N}), wherein “I” represents an ionic liquid and “N” represents an integer from one to fifty, wherein the ionic liquid system comprises from about 0.1% w/w to 100% w/w, based on the total system weight, of at least one ionic liquid comprising a cation and an anion, wherein the conjugate acid of the anion is a perfume raw material with a pKₐ from about 0 to about 14; and wherein the ionic liquid system comprises from about 0% w/w to about 99.9% w/w, based on the total system weight, of at least one ionic liquid comprising anions whose conjugate acids are not perfume raw materials.

13. A consumer product comprising from about 0.0001% w/w to 100% w/w, based on total consumer product weight, of an ionic liquid system according to claim 1.

14. The consumer product according to claim 13, wherein the consumer product is a composition intended for the treatment of hard surfaces, soft surfaces, skin or hair.

15. The consumer product according to claim 13, wherein a 10% solution in water of the consumer product has a pH of 7 or higher.

16. A detergent composition comprising:
(a) from about 0.001% w/w to about 10% w/w, based on the total weight of the detergent composition, of an ionic liquid system made of at least one ionic liquid comprising:
(i) an anion, wherein the conjugate acid of the anion is a perfume raw material with a pKₐ of from about 0 to about 14; and
(ii) a cation selected from the group consisting of: 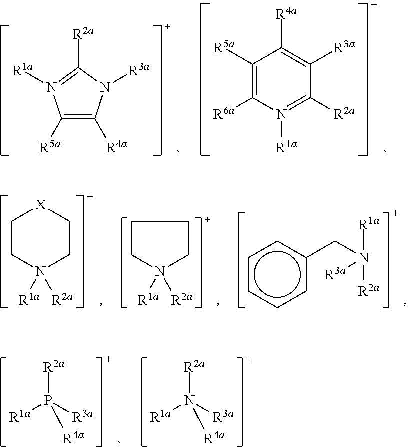
and combinations thereof
X is CH₂ or O;
each R^{1a}, R^{3a}, and R^{4a} are independently selected from hydrogen, C₁-C₂₀ alkyl, C₁-C₂₀ alkenyl, C₁-C₂₀ alkynyl, C₁-C₂₀ alkoxy, C₁-C₂₀ alkoxyC₁-C₂₀alkyl, C₃-C₇ cycloalkyl, C₃-C₇ cycloalkylC₁-C₄alkyl, C₂-C₂₀ heterocyclyl, C₆-C₁₀ aryl, C₆-C₁₀ arylC₁-C₁₀alkyl, C₁-C₁₀heteroaryl, halo, haloC₁-C₂₀alkyl, hydroxyl, hydroxyC₁-C₂₀alkyl, or —N(R^{6a})_{2;}
each R^{2a} is independently selected from hydrogen, C₁-C₂₀ alkyl, C₁-C₂₀ alkenyl, or C₁-C₂₀ alkynyl;
each R^{5a} is independently selected from hydrogen, C₁-C₂₀ alkyl, C₁-C₂₀ alkenyl, C₁-C₂₀ alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, alkylene chain, alkenylene chain, or alkynylene chain; and
each R^{8a} is independently selected from a hydrogen, alkylene chain, alkenylene chain, or alkynylene chain.

17. The detergent composition according to claim 16, further comprising: (b) from about 1% w/w to about 50% w/w, based on the total weight of the detergent composition, of a detersive surfactant.

18. The detergent composition according to claim 16, wherein the ionic liquid system is provided in a perfume microcapsule, which optionally further comprises one or more perfume raw materials.

19. A method of delivering and/or depositing fragrance onto a fabric comprising the steps of optionally washing and/or rinsing the fabric, contacting the fabric with a detergent composition according to claim 16, then optionally washing and/or rinsing the fabric.

20. A method of controlling malodour comprising contacting a substrate comprising a malodour with a composition selected from the group consisting of the composition of the consumer product of claim 13, the detergent composition of claim 16 and mixtures thereof.
